(19) United States
(12) Patent Application Publication
(10) Pub. No.: US 2011/0040234 A1
(43) Pub. Date: Feb. 17, 2011

(54) DILATOR AND TAMPON APPLICATOR DEVICE

(76) Inventor: Bernard CHAFFRINGEON, Bulach (CH)

Correspondence Address: OLIFF & BERRIDGE, PLC, P.O. BOX 320850, ALEXANDRIA, VA, 22320-4850, US

(21) Appl. No.: 12/856,136

(22) Filed: Aug. 13, 2010

Related U.S. Application Data
(60) Provisional application No. 61/272,095, filed on Aug. 14, 2009.
(60) Provisional application No. 61/272,678, filed on Oct. 20, 2009.
(60) Provisional application No. 61/258,859, filed on Nov. 6, 2009.
(60) Provisional application No. 61/282,987, filed on May 3, 2010.
(60) Provisional application No. 61/344,346, filed on Jul. 1, 2010.

(51) Int. Cl. A61F 13/20

(52) U.S. Cl. 604/15; 604/385.17

(57) ABSTRACT

A tampon applicator having an annular gripping element and a dilating element connected to the annular gripping element. The dilating element has a plurality of longitudinal members delimited by a plurality of longitudinal slots. The dilating element is configured with a conical section and a neck section, the conical section being between the annular gripping element and the neck section.

This nonprovisional application claims the benefit of U.S. Provisional Application No. 61/272,095, filed Aug. 14, 2009; U.S. Provisional Application No. 61/272,678, filed Oct. 20, 2009; U.S. Provisional Application No. 61/258,859, filed Nov. 6, 2009; U.S. Provisional Application No. 61/282,987, filed May 3, 2010; and U.S. Provisional Application No. 61/344,346, filed Jul. 1, 2010.

### BACKGROUND

This disclosure relates to the field of period and/or hygienic tampons and to devices that absorb natural body effluents, such as menstruation flow and cervical mucus.

Period and/or hygienic tampons (hereinafter “tampons”) are made of absorbent material, for example, in the form of sheets and/or strips that are folded and/or wrapped by techniques known in the art for making tampons, which are configured to be placed inside the vagina. Tampons are commonly used no matter the volume of secretions to be absorbed, and are generally configured to retain a median or even high flow, so that the tampons are suitable for most users.

Some users take direct hold of the tampon and introduce it into the vaginal cavity, while others prefer to perform intromission of the tampon with an applicator. When a tampon is intromissed without an applicator, the intromission may be painful for the user because, due to the relatively large cross-section of the tampon with regard to the introducing orifice and the relatively rough surface of the tampon at the periphery, intromission of the tampon may cause lesions or irritations by friction of the vaginal wall, especially in the area of muscular narrowing of the vaginal wall. In addition to the unpleasant feeling of pain resulting from the occurrence of such lesions or irritations, the lesions or irritations increase the risk of contracting an infectious disease, such as a sexually transmissible infectious disease.

When an applicator is used to intromiss a tampon, the user takes hold of an assembly comprising an applicator made in the form of a tube into which a tampon has been slipped, and a plunger is inserted into the tube that is intended to be used to eject the tampon from the applicator. In use, some users will press the assembly against the genital organ so that the distal end of the tube remains near the introducing orifice of the genital organ. In this case, the disadvantages encountered are the same as those during intromission without an applicator, discussed previously. Alternatively, some users will introduce the applicator deeper until reaching the vaginal cavity. In this case, the risk of the tampon rubbing against the vaginal wall will be minimized. However, the user will still be confronted with the unpleasant feeling of deeply introducing an object having a diameter substantially equal to that of the tampon.

### SUMMARY

Disclosed herein are tampon applicator devices configured to eliminate all or part of the disadvantages mentioned before. The devices are configured to operate as a dilator and a tampon applicator. They comprise a body having at least one proximal section provided with a proximal end area by which a ready-to-use hygienic tampon can be introduced, and a distal section intended to be introduced into a user's vaginal cavity, the distal section being configured so that:
it can change from an initial resting state, in which it delimits a longitudinal interior passage, the cross-section of which is closed or smaller than the cross-section of the tampon, to a deployed state, in which the cross-section of the interior passage opens substantially radially and adapts to the cross-section of the tampon, so as to allow for longitudinal movement of the tampon through the interior passage, and
it is long enough to allow for release of the tampon directly into the vaginal cavity, without the tampon making prolonged contact with the area of the muscular narrowing of the vaginal wall during intromission of the tampon.

Thus, the possibility of rubbing of the tampon against the vaginal wall, namely within an area of muscular narrowing around the introducing orifice, is reduced or eliminated due to the sleeve-type distal section of the applicator being between the muscular narrowing around the introducing orifice and the tampon during intromission, and due to the sleeve-type distal section emerging directly into the vaginal cavity. Thereby, any risk of lesion or irritation is reduced or eliminated.

Also, insertion of the dilator is easy and atraumatic due to the fact that in the initial resting state, the cross-sectional diameter of the distal section is much smaller than the cross-sectional diameter of a tampon. Consequently, the distal section may be slipped through the introducing orifice of the vaginal wall with minimal or no physical pain or psychological stress. This smaller cross-sectional diameter, in combination with adequate length, provides for flexibility of the distal section in a plane perpendicular to the axis of introduction, so that the distal section is capable of deforming to adapt to the anatomical shape of the vaginal cavity. This is an important advantage especially for the population of young users, not yet familiar with the intromission of tampons, and who do not perfectly control the angle of introduction to allow for easy insertion of the tampon into the vaginal cavity.

The applicators may be designed to adapt to any size, shape, and type of tampon. The applicators may be configured to allow the intromission of tampons of different sizes and diameters with the same applicator.

Because the applicators are configured to minimize contact of the tampon with the vaginal wall during intromission, the benefits of using tampons having a profiled distal end so as to minimize the unpleasant feeling perceived by the user are cancelled out when intromissing such tampons with applicators according to this disclosure. Indeed, when using an applicator according to the disclosure, the feeling perceived by the user will be the same regardless of the profile of the tampon's distal end. It is thus possible, or even desirable, to use tampons having a more flared distal end and that are less compressed than conventional tampons, to allow for faster and more efficient absorption of the natural effluents making contact with the distal end.

As will be described herein, the applicators may be configured to allow a user to slide the tampon into the dilator with a finger, or with a plunger.

### BRIEF DESCRIPTION OF THE DRAWINGS

The implementation of embodiments of the invention will be better understood by means of the detailed description given below with reference to the drawings, wherein:

FIG. 1A is a side view of a dilator according to the disclosure;

FIG. 1B is a side view of the dilator of FIG. 1 in a deployed position;

FIG. 2 is a perspective view of the dilator of FIG. 1;

FIGS. 3 and 4 are perspective views of dilators configured with different shaped annular gripping elements;

FIG. 5 is a side view of a dilator with additional structural elements;

FIG. 6 is a detail view of the end of the distal section of the dilator represented in FIG. 5;

FIG. 7 is a partially enlarged longitudinal sectional view of a dilator having an interior surface configured with a series of internal shoulders;

FIGS. 8a and 8b are longitudinal sectional views of the dilator depicted in FIG. 7 in the deployed position, with and without a tampon, respectively;

FIGS. 9 to 12 are perspective views, some of them partial, of a dilator fitted with a guiding device, before and after operation;

FIGS. 13 and 14 are perspective views of the guiding device represented in FIGS. 9 to 12 configured with cutouts;

FIG. 15 is a perspective view of a dilator fitted with another guiding device;

FIGS. 16-20 are side views, some of them partial, of the applicator depicted in FIG. 15, in various stages of deployment;

FIG. 21 is an enlarged side view of an assembly comprising the applicator represented in FIG. 4 fitted with a cover element;

FIG. 22 is a perspective view of the assembly represented in FIG. 21;

FIG. 23 is a front view of the assembly represented in FIG. 21;

FIG. 24 is a top view of another cover element that may be fitted to a dilator;

FIGS. 25 and 26 are schematic longitudinal sectional views of a dilator fitted with another cover element, before and after the cover element is opened;

FIG. 27 is a schematic longitudinal sectional view of the dilator fitted with the cover element depicted in FIG. 25, where the cover element further includes a pushing member;

FIG. 28 is a perspective view of a dilator configured with a tube and a pushing member;

FIG. 29 is a side view of the assembly represented in FIG. 28;

FIG. 30 is a longitudinal sectional view of the assembly represented in FIG. 29;

FIG. 31 is a perspective enlarged view of the pushing member of the assembly represented in FIG. 28;

FIG. 32 is a detail view of the end of the pushing member represented in FIG. 31;

FIG. 33 is a perspective view of the ring inserted in the assembly represented in FIG. 28;

FIG. 34 is a sectional enlarged view along line X-X of the ring represented in FIG. 33;

FIG. 35 is a side view of the assembly represented in FIG. 28 once the pushing member has been armed;

FIG. 36 is a longitudinal sectional detail view of the assembly represented in FIG. 35;

FIG. 37 is a side view of the assembly represented in FIG. 28 at the end of the operation;

FIG. 38 is a side view of a dilator configured with a tube and another pushing member;

FIG. 39 is a perspective enlarged view of the pushing member of the assembly represented in FIG. 38;

FIG. 40 is a side view of the assembly represented in FIG. 38 at the end of the operation;

FIGS. 41 and 42 respectively are a side view and a longitudinal sectional view of another assembly according to the disclosure, ready for use;

FIGS. 43 and 44 respectively are a side view and a longitudinal sectional view of the assembly represented in FIGS. 41 and 42 after the pushing member has been armed;

FIGS. 45 and 46 respectively are a side view and a longitudinal sectional view of the assembly represented in FIGS. 41 and 42 when the tampon is being extracted;

FIGS. 47 and 48 respectively are a side view and a longitudinal sectional view of the assembly represented in FIGS. 41 and 42 at the end of the operation;

FIG. 49 is a perspective view of another assembly according to the disclosure;

FIG. 50 is a perspective view of the applicator of the assembly represented in FIG. 49;

FIG. 51 is a perspective view of the pushing member of the assembly represented in FIG. 49;

FIG. 52 is a perspective view of the assembly represented in FIG. 49 after the pushing member has been armed;

FIG. 53 is a schematic longitudinal sectional view of a dilator fitted with a tube having a tapered section;

FIGS. 54 to 59 are a side view and a perspective view of three specific kinds of tampons, which can be used with the assembly represented in FIG. 53;

FIGS. 60 to 62 are enlarged side views of another applicator according to the disclosure, respectively before, during, and at the end of the operation; and

FIGS. 63 and 64 are longitudinal sectional views of another assembly according to the disclosure.

### DETAILED DESCRIPTION OF EMBODIMENTS

In the following detailed description of the figures described above, the same elements or elements fulfilling identical or similar functions may keep the same reference numerals to facilitate the understanding of the disclosure.

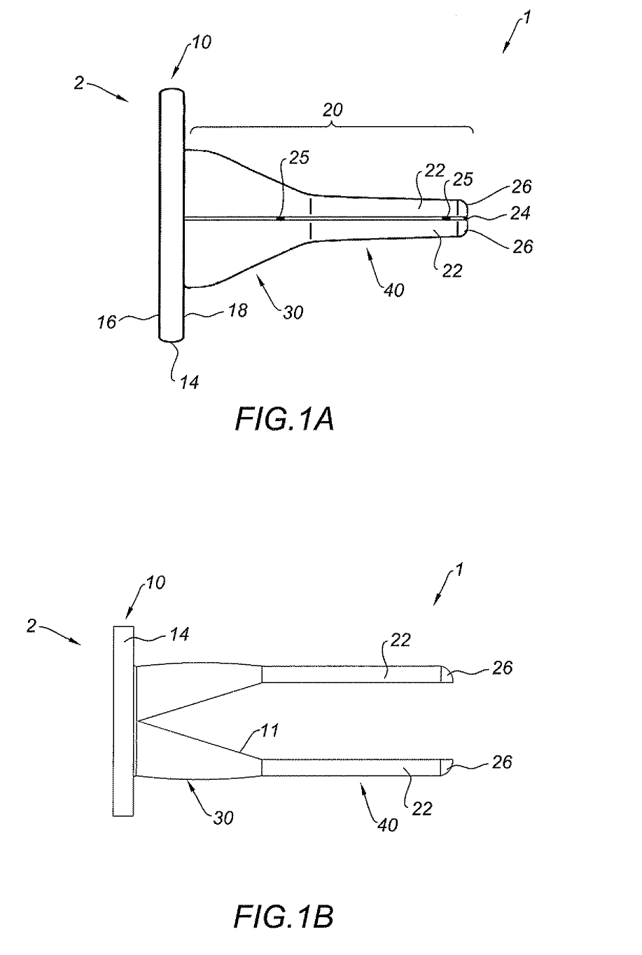

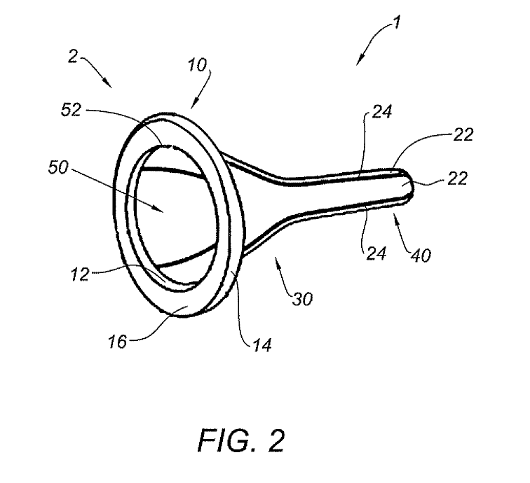

An applicator 1, such as represented in FIGS. 1 and 2, comprises a dilator 2 having an annular gripping element 10 and a dilating element 20 connected to the annular gripping element 10. The dilating element 20 comprises a plurality of longitudinal members 22 delimited by a plurality of longitudinal slots 24. The dilating element 20 has a conical section 30 and a neck section 40, the conical section 30 being between the annular gripping element 10 and the neck section 40. For convenience, the end of the dilator 2 where the annular gripping element 10 is situated will be referred to herein as the proximal end, and the end of the dilator 2 where the neck section is situated will be referred to herein as the distal end.

The various components of the applicator 1 discussed above, and as described herein, may independently or collectively be formed from a variety of materials, including, for example, plastic, including biodegradable plastic; metal; paper; cardboard; ceramic; glass; and mixtures thereof, unless otherwise specified.

The annular gripping element 10 comprises an inner periphery 12, an outer periphery 14, a first major surface 16, and a second major surface 18. The inner periphery 12 defines opening 52, which may be of any size and shape suitable for introducing a tampon into the dilator 2. For example, the shape of the opening 52 may be substantially round, such as the shape of a circle or an oval; may have a polygonal shape; or may be any irregular shape comprising curved and/or straight lines.

The opening defined by the inner periphery of the annular gripping element may have an average diameter, for example, in the range of from about 0.5 to about 10 cm, such as from about 0.5 to about 6 cm, or from about 3 to about 10 cm, or from about 1 to about 3 cm, or from about 1.5 to about 2.5 cm. In some embodiments, the average diameter of the opening is about 1.1 cm, 1.2 cm, 1.3 cm, 1.4 cm, 1.5 cm, 1.6 cm, 1.7 cm, 1.8 cm, 1.9 cm, 2.0 cm, 2.1 cm, 2.2 cm, 2.3 cm, 2.4 cm, 2.5 cm, 2.6 cm, 2.7 cm, 2.8 cm, 2.9 cm, or 3.0 cm.

The opening may have an area, for example, in the range of from about 0.20 to about 80 cm², such as from about 0.20 to about 30 cm², or from about 25 to about 80 cm², or from about 0.80 to about 7 cm², or from about 1.75 to about 5 cm², or from about 2.25 to about 4.5 cm².

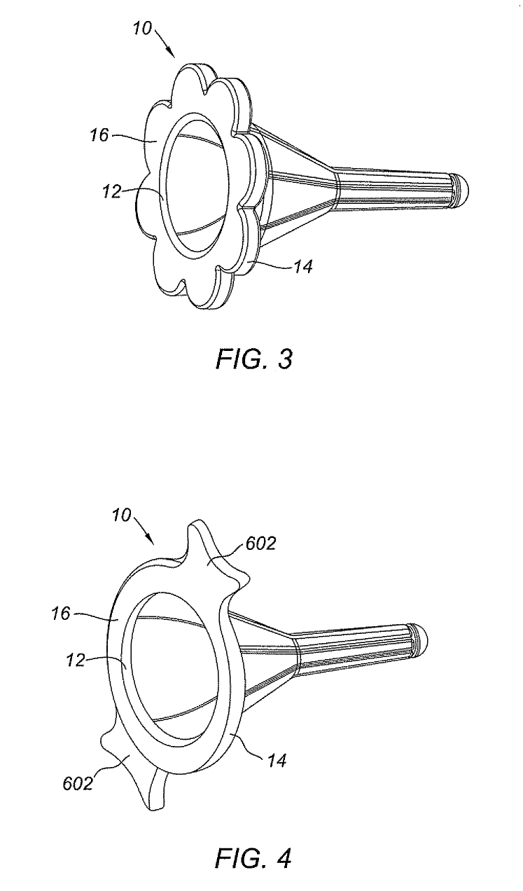

The outer periphery 14 of annular gripping element 10 depicted in FIG. 2 is circular in shape. However, the outer periphery may be configured to have any desired shape. For example, the outer periphery 14 may also be configured for aesthetic purposes, such as to give the dilator 2 a flower-like appearance, as depicted in FIG. 3. As depicted in FIG. 4, annular gripping element 10 may be configured to have lugs 602 oriented perpendicularly to the axis of the dilator 2. Although two lugs are shown, the annular gripping element may be configured with a single lug, or with more than two lugs. Additionally, the lugs may be positioned in other configurations where the lugs are not diametrically opposed and/or oriented perpendicularly to the axis of the dilator. The one or more lugs may function, for example, to enhance gripping of the annular gripping element, or may be configured to serve as a point of attachment for other structural elements, such as cover element 605 depicted in FIG. 22.

The annular gripping element 10 has a first major surface 16 facing the proximal end of dilator 2. The second major surface 18 of annular gripping element 10 opposes the first major surface 16, and faces towards the distal end of dilator 2. The first major surface 16 and second major surface 18 may be planar or substantially planar in a direction that is perpendicular or substantially perpendicular to the longitudinal direction of interior passageway 50. Alternatively, the first and second major surfaces 16 and 18 may be angled in a direction either towards or away from the distal end of dilator 2, and/or may not be planar.

The annular gripping element 10 may, for example, have an average thickness from the first major surface 16 to the second major surface 18 in a range of from about 1 to about 10 mm, such as from about 1 to about 6 mm, or from about 4 to about 10 mm, or from about 2 to about 4 mm. The thickness may be uniform, substantially uniform, or variable throughout the annular gripping element 10.

The dilator 2 has an interior passageway 50 that runs in a longitudinal direction of the dilator 2 beginning at interior passageway opening 52 and continues into the conical section 30. The interior passageway may truncate in the conical section, or it may continue into the neck section.

The dilator is configured so that, for example, the distal end of a tampon may be inserted into the interior passageway by way of opening 52. However, the passageway is preferably configured to impede the movement of the tampon towards the distal end of the dilator. This may be done, for example, by narrowing or truncating the passageway in a manner so that, after traveling some distance through the passageway towards the distal end of the dilator, some portion of the tampon comes into contact with the interior structure of the dilator. As force is applied to the tampon in a direction towards the distal end of the dilator, the tampon pushes against the interior structure of the dilator. When sufficient force is applied, the longitudinal members radially spread apart, expanding the passageway, and the tampon is able to continue its movement towards the distal end of the dilator, and is eventually expelled from the distal end of the dilator.

Two or more longitudinal slots 24 run in a longitudinal direction along at least a portion of the conical section 30 and the neck section 40 of the dilator 2, and are distributed around the periphery of the dilator 2, thus delimiting two or more longitudinal members 22 configured to have a radial spreading elasticity adapted to the cross-section of a tampon. The slots 24 may be uniformly or non-uniformly distributed around the periphery of the dilator 2. In FIG. 1A, slot 24 is depicted as running from tips 26 to annular gripping element 10. However, the dilator may be configured with slots that end at some portion of the dilating element short of reaching the annular gripping element, such as, for example, at a point located in the conical section or between the conical section and the gripping element.

Adjacent longitudinal members 22 may be connected to each other by one or more junction points 25 for enabling calibrated opening of the longitudinal members 22. The junction points 25 connect the longitudinal members 22 together before use. As represented schematically in FIG. 1, each longitudinal member 22 may initially be attached to each of the two adjacent longitudinal members 22, for example, by two junction points 25, a first one located in the conical section 30, and a second one in the neck section 40. The junction points 25 may be made integrally from the same material as the longitudinal members 22.

Due to these junction points 25, the radial opening of the longitudinal members 22 is calibrated. In operation, the user thus starts by introducing a tampon 3 into the interior passageway 50 of dilator 2 by way of opening 52, then takes hold of the dilator 2 by positioning, for example, her forefinger and her middle finger in abutment against the second major surface 18 of annular gripping element 10. Before, while, or preferably after neck section 40 is introduced into the vagina so as to emerge directly into the vaginal cavity where the tampon 3 is to be delivered, the user may exert force on the proximal end of the tampon 3 by means of, for example, her thumb or a plunger, so as to cause the distal end of tampon 3 to slide along the interior passageway towards and into the conical section 30 until the distal end of tampon 3 abuts against the narrowing walls defining the conical section 30. Thus, the distal end of the tampon transfers this force to the longitudinal walls in contact with the tampon. When sufficient force is applied to the tampon, this successively causes rupturing of the junction points 25 located in the conical section 30, then rupturing of the junction points 25 located in the neck section 40. It should be understood that it may be of advantage to reinforce the resistance of the junction points 25 arranged in the neck section 40 to allow for real propulsion of the tampon 3 in the direction of the vaginal cavity once these junction points 25 will have given in.

The longitudinal members 22 can be made to deform by flexion in directions perpendicular to the axis of introduction so as to adapt the dilator to the anatomical shape of the vaginal cavity. This will further reduce the feeling of discomfort that might be perceived by the user.

By exerting sufficient force on the proximal end of the tampon 3, for example with the thumb, while performing counter-pressure by positioning, for example, the forefinger and the middle finger around the gripping element 10, sliding of the tampon 3 will then successively cause the conical section 30 and neck section 40 of the longitudinal members 22 to spread apart radially. The radially spreading apart of the longitudinal members 22 thus may exert force against the muscular narrowing of the vaginal wall and dilate the muscular narrowing to allow passage of the tampon past the muscular narrowing with minimal or no direct contact between the tampon and the muscular narrowing.

Insertion of the dilator into the vaginal opening is easy and minimally traumatic due to the fact that in the initial resting state, the outer cross-sectional diameter of the neck section 40 is much smaller than the cross-sectional diameter of a tampon. Consequently, the neck section 40 may be slipped through the introducing orifice of the vaginal wall with minimal or no physical pain or psychological stress. This smaller cross-sectional diameter, in combination with adequate length, also may provide for flexibility of the neck section 40 in a direction perpendicular to the axis of introduction, so that the neck section 40 is capable of deforming for adapting to the anatomical shape of the vaginal cavity.

The neck section of the dilator may be generally described as the section comprising the distal end of the dilator. The neck section may have a substantially uniform diameter throughout the length of the neck section, or it may have sections of changing, preferably increasing, cross-sectional areas in a direction heading towards the proximal end of the dilator.

The neck section 40 may, for example, have a length in the range of from about 0.5 to about 10 cm, such as from about 0.5 to about 5 cm, or from about 4 to about 10 cm, or from about 1 to about 4 cm, or from about 1.5 cm to about 3.0 cm. As used herein, the length of the neck section refers to the shortest distance between the proximal end 31 and the distal end 32 of neck section 40.

As can be seen in FIG. 1, at least a portion of the exterior surface of the conical section 30 is configured in a tapered shape in a direction going from the annular gripping member 10 towards the neck section 40. The tapering may be continuous and substantially uniform, or may be staged. This tapered shape allows the user to properly position the dilator in the muscular narrowing of the vagina and to initiate some opening of that area of the vagina wall. The outer surface of at least that portion, and preferably all, of the applicator is preferably comprised of a low friction material to minimize discomfort to the user.

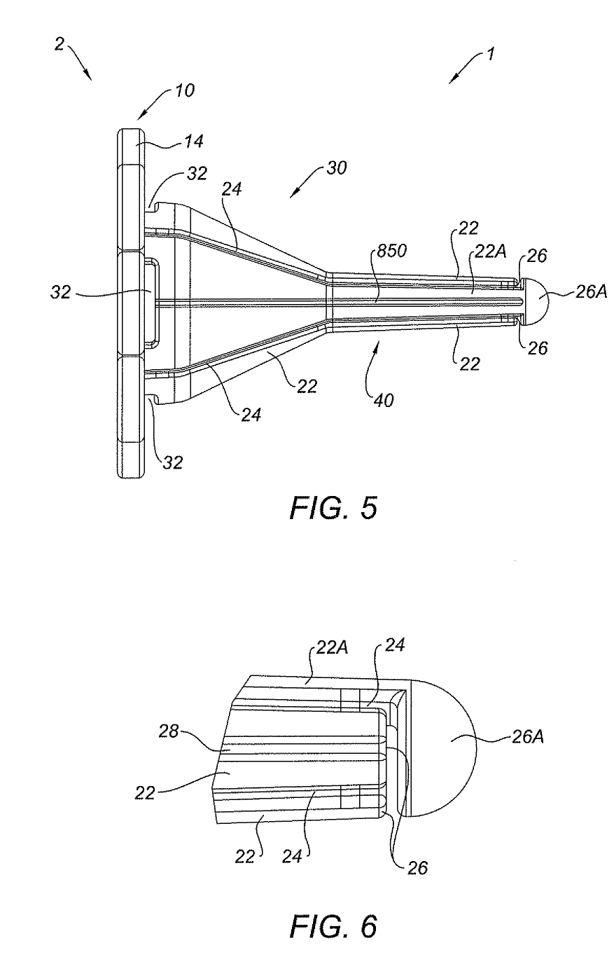

The longitudinal members 22 may be substantially equal in length or otherwise configured so that the tips 26 of the longitudinal members 22 are flush or substantially flush with each other, such as shown in FIG. 1. The tips 26 may be smoothed and/or rounded to eliminate sharp edges. Alternatively, referring to FIGS. 5 and 6, longitudinal member 22a may have an extended tip 26a in the shape of a semispherical body oriented so as to cover, in a closed position, tips 26 of the other longitudinal members 22. As shown in FIG. 6, these other tips 26 are not attached to the semispherical body of extended tip 26a. However, one or more of the other tips may be detachably attached to the semispherical body by, for example, a junction point.

The convex profile of the semispherical body of tip 26a assists in centering and guiding the neck of the dilator into the vagina, which may be particularly advantageous to young users who are not yet familiar with how to intromiss a tampon. Also, because the semispherical body covers the ends of the other longitudinal members, these other ends are less likely to cause injury or irritation to the user upon insertion of the dilator.

Referring to FIG. 5, one or more longitudinal members 22 may have at least one longitudinal groove 28 formed in the outer surface of longitudinal member 22. The longitudinal grooves 28 reduce the surface area of the dilator in contact with the vaginal wall, thus further facilitating insertion of the dilator into the vaginal cavity, and improving comfort of use. Although grooves are depicted, it should be understood that these surface cavities could alternatively be made in any other size and shape.

Longitudinal member 22 may be configured with a window 32 arranged in the conical section 30. The windows 32 may preferably be uniformly distributed around a circumference of the conical section. The windows 32 may be configured to decrease the amount of force needed to radially spread the longitudinal members 22, and thus allow for better control of the opening of the longitudinal members 22.

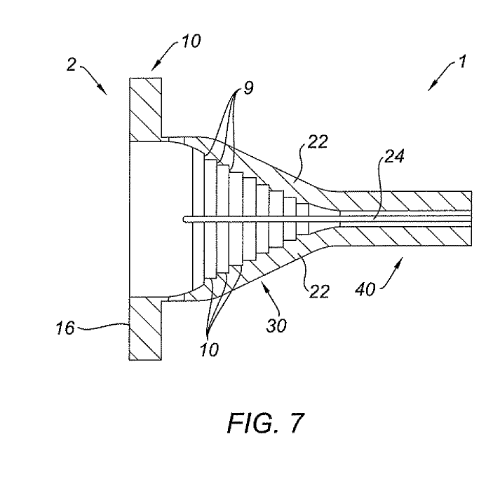

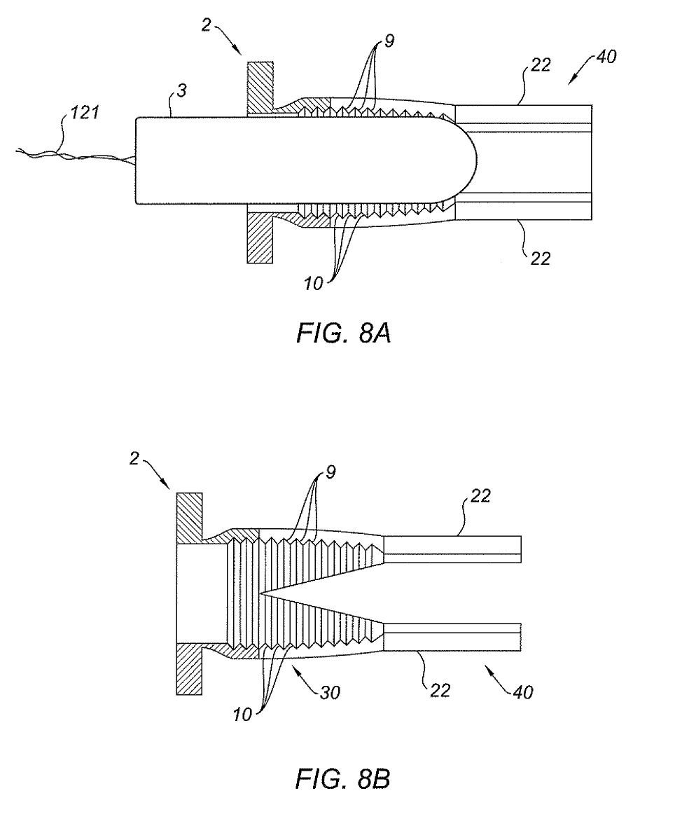

FIG. 7 depicts a dilator 2 where the interior surface of conical section 30 is configured with a series of internal shoulders 34 each having an edge 36 forming an area of rugosity. When the tampon is introduced into the passageway defined by conical section 30, but before the longitudinal members 22 have been sufficiently radially spread, the edges 36 fray and/or flare the distal end of the tampon as force exerted on the tampon causes the distal end of the tampon to exert force against the edges 36. This fraying/flaring of the distal end of the tampon enables faster and more efficient absorption of the natural effluents making contact with the distal end of the tampon 3 once inserted into the vaginal cavity. Although edges 36 initially impede movement of the tampon through the conical section 30, once junction points 25 are ruptured, longitudinal members 22 are radially spread by the tampon to a point where the edges 36 are in a position relative to the surface of the tampon to afford minimal contact area between the tampon 3 and edges 36, as schematically depicted in FIG. 8. Thus, when the longitudinal members 22 are fully radially spread by the tampon, edges 36 offer minimal resistance to the sliding of the tampon 3, and the side surface of the tampon is preserved from any untimely deterioration.

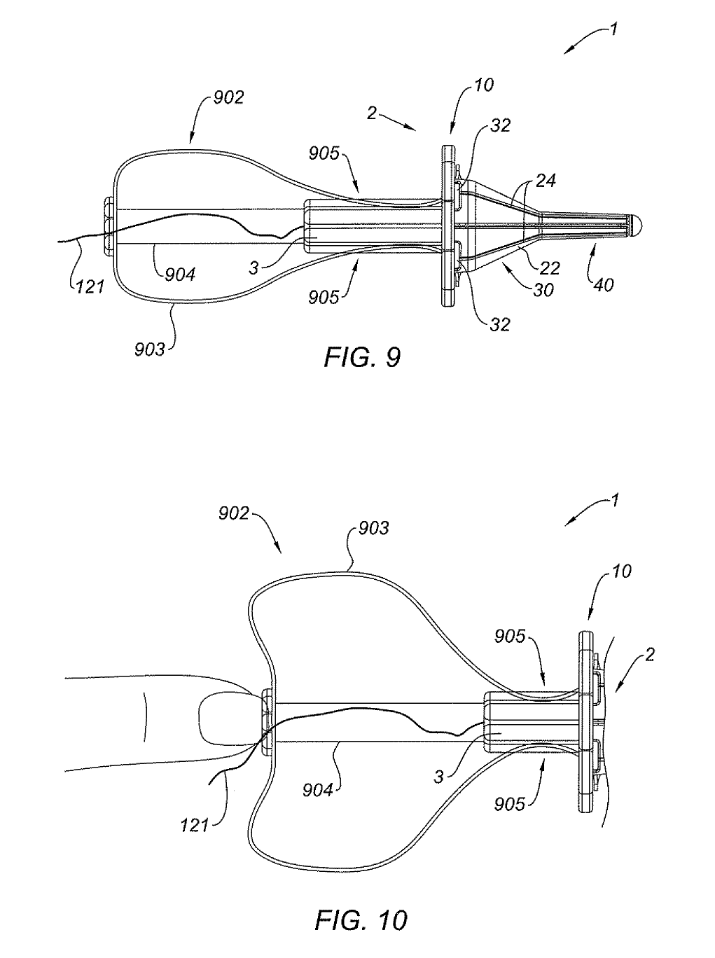

The applicator may further comprise a guiding device attached to the dilator. Referring to FIGS. 9 to 13, the guiding device 902 may comprise a blade spring loop 903 and a pushing member 904. The loop may be fashioned from a band-shaped piece of resilient material, the end portions of which thread through and are affixed to two of the windows 32, although attachment of the blade spring loop 903 to the dilator 2 is not limited to this configuration. Indeed, the blade spring loop 903 may be attached to the dilator 2 at, on, or through any portion of the dilator 2, including the annular gripping element 10 and the conical section 30. Also, the points of attachment of the blade spring loop need not be located at or near the ends of the blade spring loop. The blade spring loop may be attached to the dilator in a manner in which it may be detachable, or it may be affixed in a more permanent manner, such as by welding or fusing. In some embodiments, the blade spring is integrally molded to the dilator during the same molding operation.

The blade spring loop may be made from any resilient material that will urge the blade spring loop to return to its original position, or substantially thereto, after deformation of the loop. The length, width, and thickness of the blade of material defining the blade spring loop are not particularly limited. Also, the overall shape of the loop is not particularly limited. In some embodiments, the proximal end of the loop is configured to be sufficiently narrow so that a user cannot fit a finger between the pushing member 904 and the inside surface of the proximal end of the loop near the point of attachment of the pushing member 904 to the blade spring loop 903 (not shown). The blade spring loop 903 may be configured to have bending areas 905 that partially enclose the tampon 3 and center the tampon in the opening of the dilator.

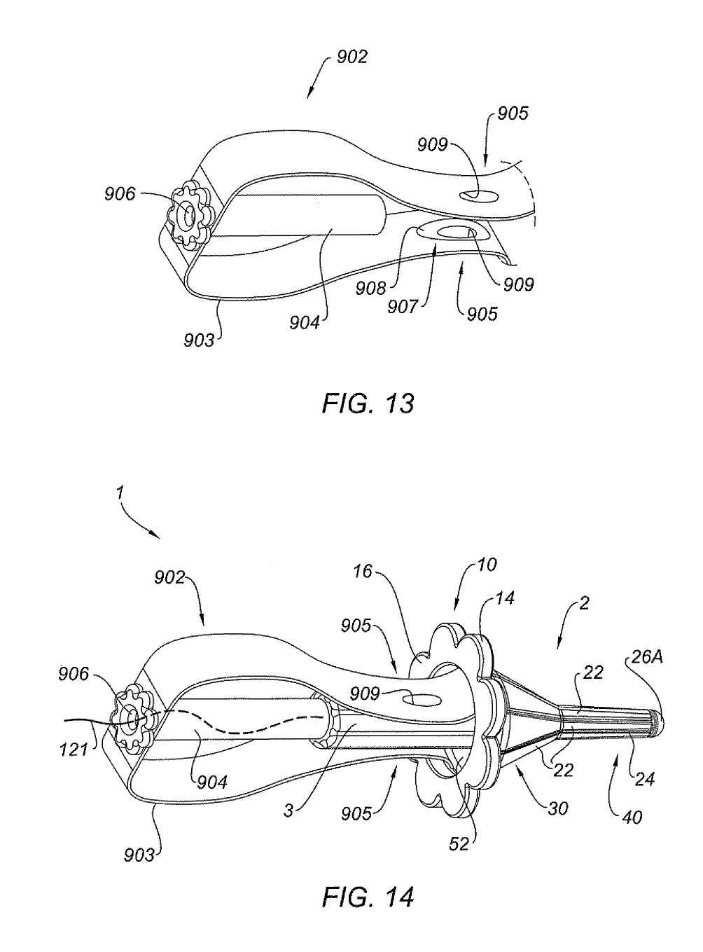

Referring to FIGS. 13 and 14, the blade spring 903 may be configured with a cutout 907 arranged in the inner surface of bending areas 905 so as to further improve the support of the tampon 3. The cutout 907 may have a concave border 908 surrounding a center through orifice 909. This configuration allows for part of the side surface of the tampon 3 to fit more securely against bending areas 905. Certain fibers composing the tampon 3 can also slightly stand out from the center orifice 909, so as to further improve “the anchoring” of the tampon 3 in a storage position.

The guiding device can be configured in a variety of shapes. As for a blade spring, it may, for instance, have a free end that is not attached to the dilator, whereby it does not form a loop with the dilator. Alternatively, the guiding device may be configured with more than one blade spring loop distributed around the circumference of the tampon 3. Alternatively, the guiding device may be configured in the shape of distinct mechanical parts made integral with the dilator by, for example, snap-fitting, ratchet motion, gluing, welding, etc.

The guiding device preferably further comprises pushing member 904 connected to the blade spring loop 903. The pushing member 904 may be attached to a portion of the blade spring loop 903 so that the ends of the pushing member 904 are aligned with the longitudinal direction of the dilator 2 and with opening 52. Although the pushing member 904 illustrated in FIGS. 9-13 is cylindrical in shape with a diameter smaller than the tampon 3, the pushing member 904 may be configured in many shapes and size. If the pushing member is configured with a size and shape that allow it to pass through the opening of the dilator, then the pushing member may be used to help push the tampon through the radially expanding extensions.

The length of the pushing member 904 is preferably substantially equal to or longer than the length of the dilator 2 to ensure that the tampon 3 is completely expelled from the dilator 2 so as to be adequately delivered into the vaginal cavity. One benefit of this configuration is that the user can avoid fluids contact with her pushing finger by preventing the finger from penetrating inside the dilator or inside the vaginal cavity. In some embodiments, the pushing member is shorter than the length of the dilator. Also, as schematically depicted in FIG. 9, the applicator 1 may be configured so that the distal end of the pushing member 904 rests on the end of the tampon 3 so as to “set” the tampon in a storage or packaging position when the applicator is bundled with a tampon.

The pushing member may be hollow or solid. As depicted in FIG. 14, pushing member 904 preferably is configured so that string 121 of the tampon 3 is slipped into the pushing member 904, and the free end thereof emerges from orifice 906.

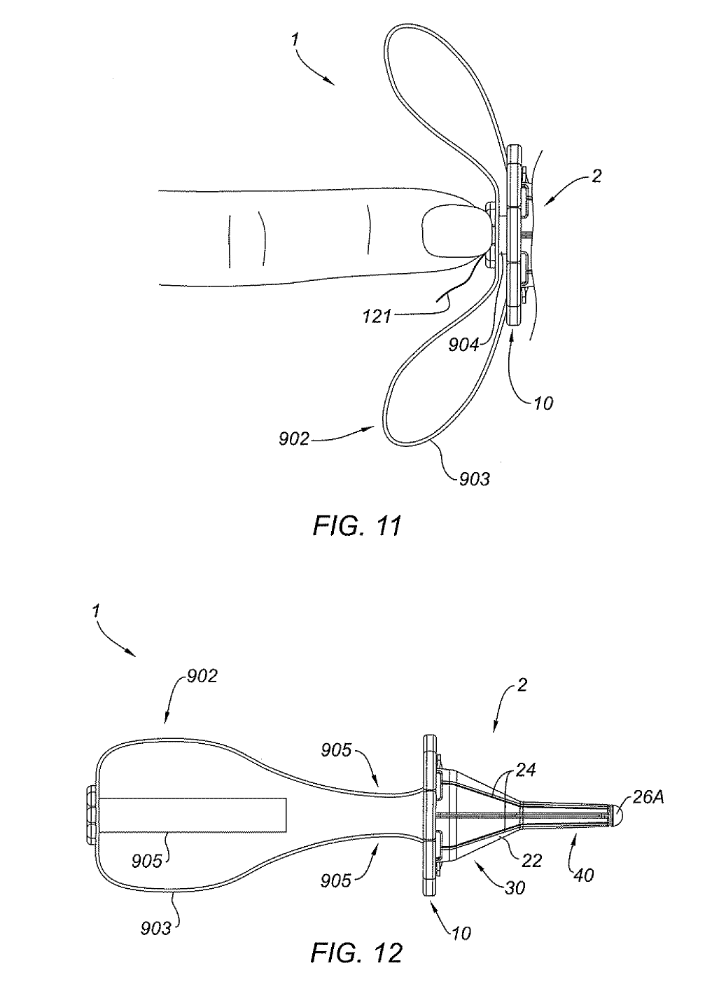

Before operation, and as represented in FIG. 9, the distal end of a tampon 3 is introduced into the proximal end of the dilator 2. Depending on the respective lengths of the tampon and the pushing member 904, the distal end of the pushing member 904 may or may not be in contact with the proximal end of tampon 3. Using, for example, a thumb or a forefinger, the user may push on the blade spring loop 903/pushing member 904 to place the distal end of the pushing member 904 in contact with the proximal end of tampon 3 and/or to maintain that contact. Intromission of the tampon into the vaginal cavity may be carried out substantially as discussed previously by applying sufficient force to the blade spring loop 903/pushing member 904 to push the tampon through the interior passageway of the dilator. As represented in FIG. 10, this pushing deforms the blade spring loop 903 towards annular gripping element 10. As depicted in FIG. 11, the guiding device 902 may be configured so that the entire or substantially entire length of the pushing member 904 can enter into the interior passageway of dilator, and the proximal end of blade spring loop 903 substantially comes into abutment against the annular gripping element 10.

When the force exerted on the blade spring loop 903 is released, the resiliency of the blade spring loop 903 causes the blade spring loop 903 to return to its original or substantially original position, thus pulling out pushing member 904 from the interior passageway of the dilator, as represented in FIG. 12. This, in turn, allows the longitudinal members 22 of the dilator 2 to return to their initial resting position as the user withdraws the dilator 2 from the vaginal opening. Thus, withdrawal of the dilator is hardly perceived by the user because of the minimal diameter of the neck section 40. The string 121 may be completely released from the dilator 2 during backward movement of the dilator.

Another substantial advantage of such a version is due to the fact that the overall mass of the assembly is minimal due to the small amount of material used for making the different components.

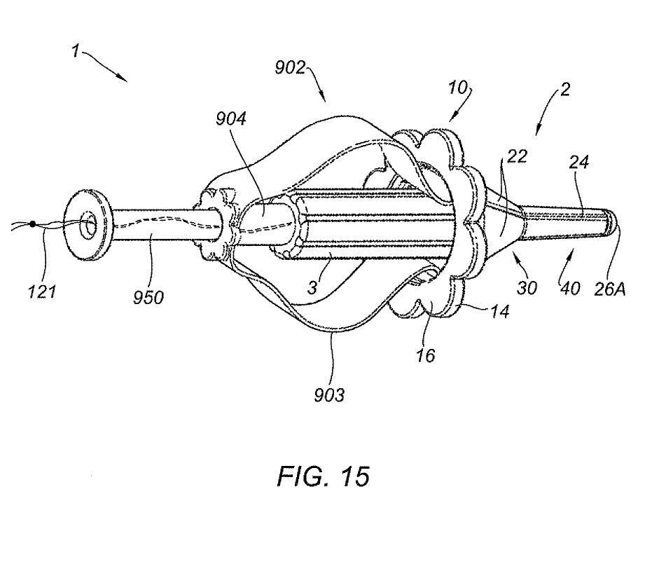

Referring to FIGS. 15 to 20, in some embodiments, the guiding device 902 may further comprise a plunger 950. In these embodiments, pushing member 904 and/or blade spring loop 903 is configured to define a passageway whereby at least a portion of plunger 950 is slidably positioned within the passageway.

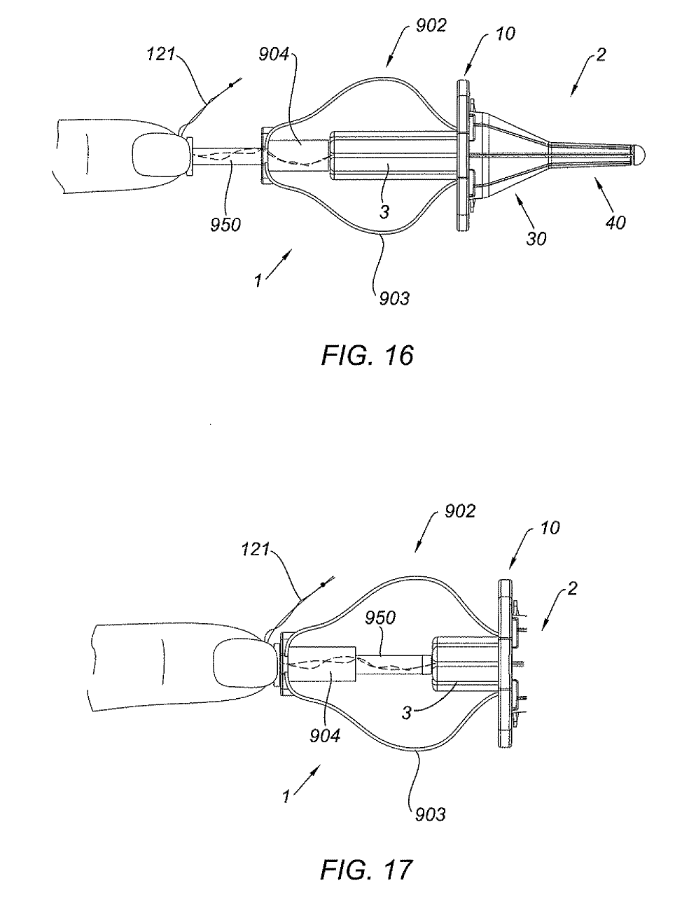

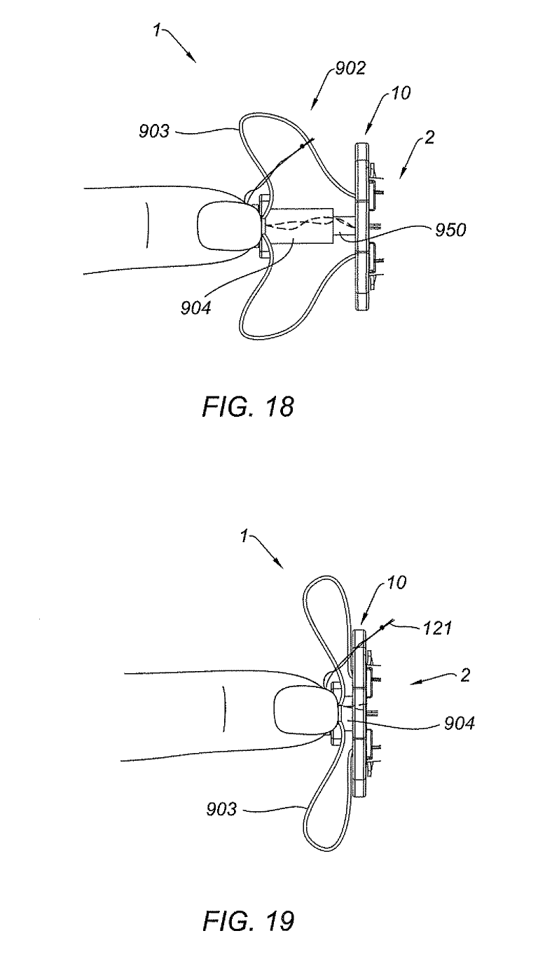

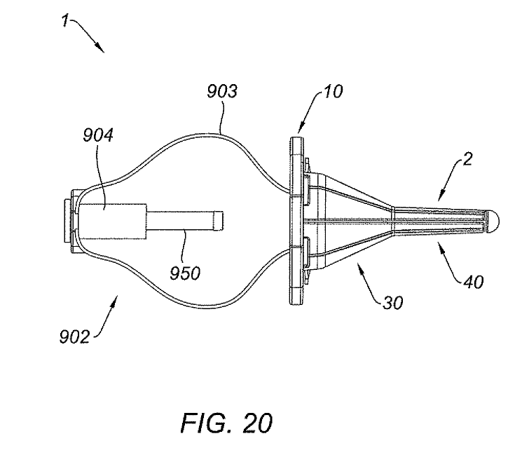

The plunger 950 may be configured so that the distal end of the plunger 950 may be pushed beyond the distal end of pushing member 904 and/or blade spring loop 903 to a distance so that the length of the pushing member 904 plus the length of the plunger 950 is sufficiently long to completely expel a tampon from the dilator 2 into the vaginal cavity. Before operation, and as represented in FIG. 16, the distal end of a tampon 3 is introduced into the proximal end of the dilator 2. The distal end of the plunger 950 is placed in contact with the proximal end of tampon 3. Using, for example, a thumb or a forefinger, the user may push on the proximal end of plunger 950 to exert a force on the proximal end of tampon 3 to begin pushing the tampon through the dilator 2. As this happens, plunger 950 slidably moves through pushing member 904 and/or blade spring loop 903 so that the proximal end of the plunger 950 moves towards the proximal end of pushing member 904 while the distal end of plunger 950 moves away from the distal end of pushing member 904. During this stage, the distal end of blade spring loop 903 may not be deformed towards annular gripping element 10. The plunger 950 continues its free movement relative to the pushing member 904 until a portion of plunger 950 engages with a portion of member 904/903, as shown in FIG. 17, so that the application of sufficient force on the proximal end of plunger 950 causes the plunger 950 and member 904/903 to move together while simultaneously pushing the proximal end of blade spring loop 903 towards annular gripping element 10, as represented in FIG. 18. As depicted in FIG. 19, the guiding device 902 may be configured so that the entire or substantially entire length of the pushing member 904 can enter into the interior passageway of dilator, and the distal end of blade spring loop 903 substantially comes into abutment against the annular gripping element 10. Upon release of the force applied to the blade spring loop 903, the resiliency of blade spring loop 903 urges the blade spring loop 903 to return to its original or substantially original position thus pulling out pushing member 904 and plunger 950 from the interior passageway of the dilator, as shown in FIG. 20, so as not to impede the return of longitudinal members 22 of the dilator 2 to their initial resting position as the user withdraws the dilator 2 from the vaginal opening.

The applicator may comprise a dilator fitted with a cover element designed to help prevent the user's pushing finger from contact with the flows from the vaginal cavity by intervening between the finger and the tampon throughout sliding of the tampon, so that the finger does not directly contact the vaginal cavity. The cover element may be woven or non-woven, plastic or non-plastic, elastic or non-elastic, impermeable or permeable.

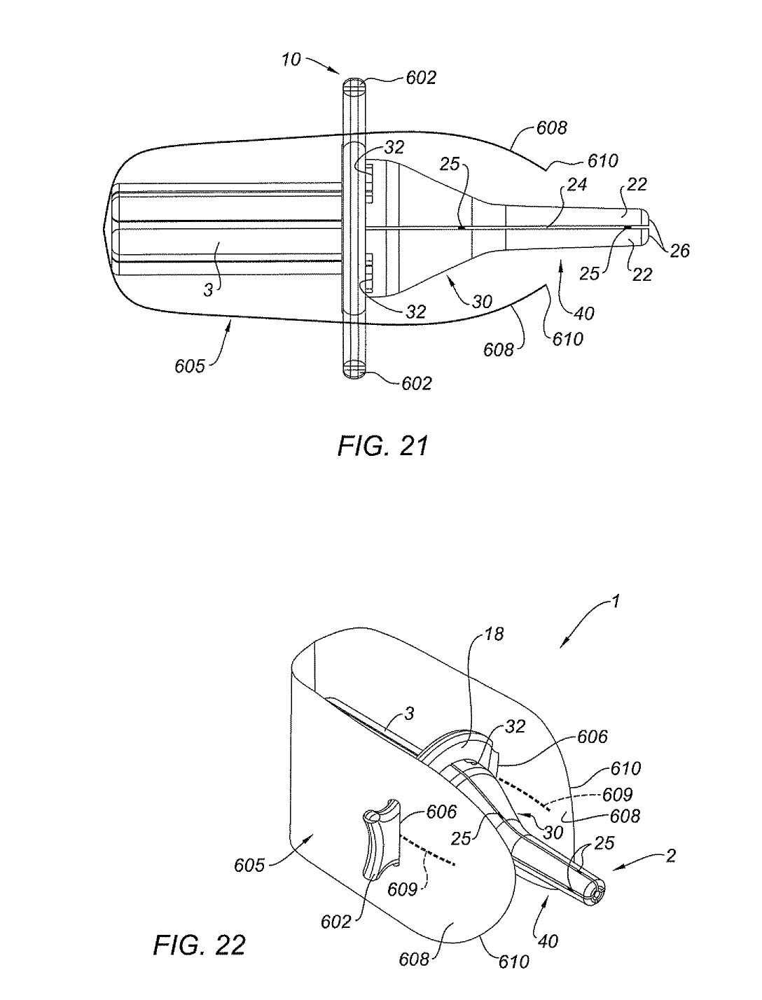

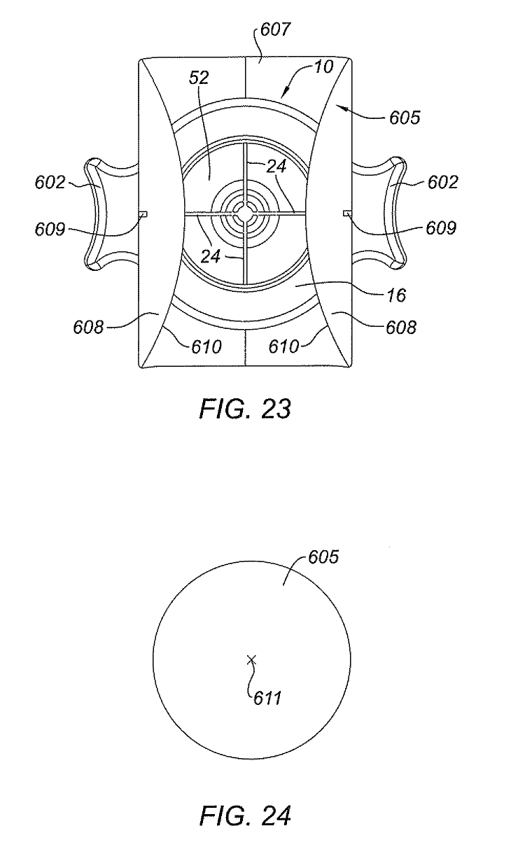

In FIGS. 21 to 23, the cover element 605 is a substantially rectangular band of material with rounded corners, having slots 606 for fastening the cover element 605 to the dilator 2. The tampon 3 is inserted into the dilator 2, as can be seen in FIGS. 21 and 22. The cover element 605 may be attached to the dilator 2 by inserting each of the lugs 602 into a slot 606 corresponding thereto. The cover element 605 may be tensioned to maintain the position of the distal end of tampon 3 in the dilator 2.

The cover element 605 may be provided with excess material 608 located between each slot 606 and the end 610 of the cover element 605 corresponding thereto, to protect the tip of the finger positioned in abutment against the corresponding lug 602, by intervening between the finger and the lips of the genital organ.

Also, each portion of the excess material 608 may be provided with a longitudinal line of weakness 609 that progressively tears upon contact with the corresponding lug 602, as the tampon 3 slides into the dilator 2, so that the cover element 605 can accompany the tampon 3 over any length of the stroke thereof until it is released into the vaginal cavity.

Alternatively, the cover element 605 may be configured as a disk as represented in FIG. 24. Here, this disk cover element 605 covers the whole tampon 3, and thereby is advantageously provided with a cross-shaped center lumen 611 for letting pass the withdrawal cord of the tampon 3. Alternatively, the cover element may be a piece of material having, for instance, a substantially square, triangular, or trapezoidal shape, or any other polygonal shape, or any other shape comprising straight and/or curved lines.

The cover element may be fastened to the annular gripping element in any manner known to those skilled in the art, including by adhesion, bonding, and/or welding.

The cover element may be coated with one or more substances, such as, for example, disinfectant for fingers, cicatrizer, lubricant, a product providing pH control, etc. Different sides and/or different portions may be coated or embedded with different substances.

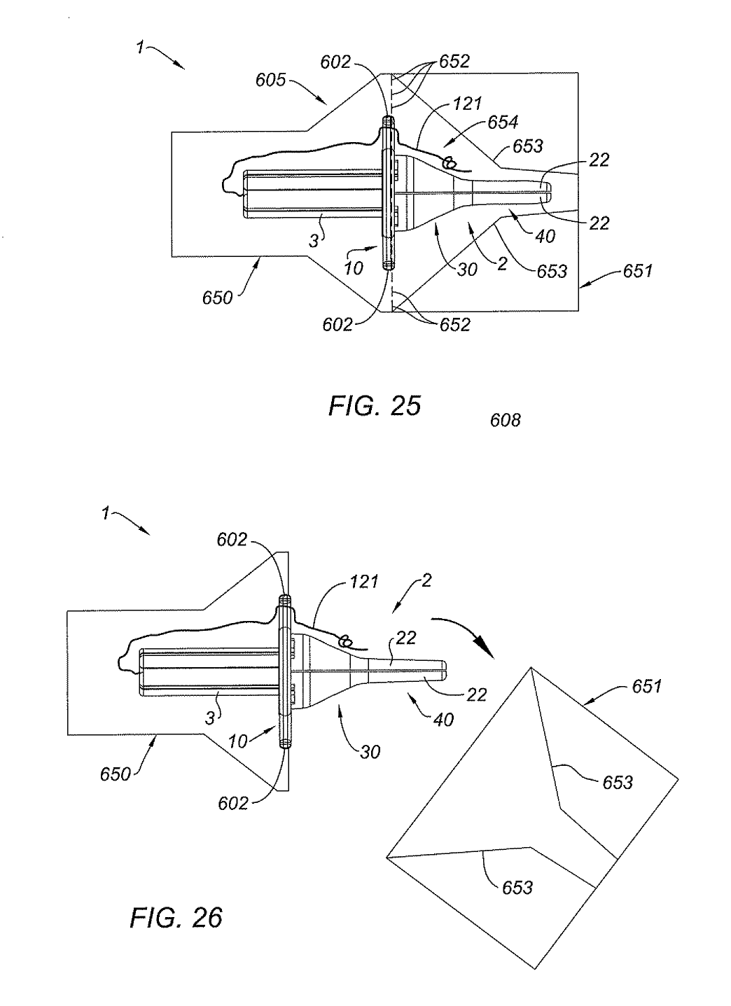

FIG. 25 depicts a dilator 2 with a tampon 3 that is enclosed in cover element 605. Cover element 605 is a sleeve of material, such as a complete packaging bag, that envelops or substantially envelops the assembly formed by the dilator 2 and the associated tampon 3. The cover element 605 has a proximal part 650 covering the tampon 3 engaged in the dilator 2, and a distal part 651 covering the dilator 2. The distal part 651 may be integral with the proximal part 650, and the cover element 605 may be configured with areas of weakness 652 so that the distal part 651 can be detached from the proximal part 650 in a predetermined fashion. In FIG. 25, the areas of weakness 652 are located substantially inside the plane of the annular gripping element. However, the areas of weakness 652 may be arranged elsewhere, such as around the conical section and/or the neck section of the dilator 2. The areas of weakness 652 may also be arranged, for example, in a transverse plane to the longitudinal members 22.

The distal part 651 may comprise partially oblique welding areas 653 that define a funnel-shaped housing 654 for the dilator 2, or be otherwise configured to fit snugly against or conform to the contours of the dilator 2. This may be done to help prohibit or impede the untimely radial spreading of the longitudinal members of the dilator 2.

A portion of the proximal part 650 of cover element 605 may be attached or otherwise secured to a portion of the dilator 2 so that the proximal part 650 remains attached to the dilator when the distal part 651 is detached from the proximal part 650. In some embodiments, this attachment is made so that an opening is at least liberated for the passage of the free end of the withdrawal cord 121 in the direction of the distal end of the dilator 2. The annular gripping element 10 may be provided with, for example, an orifice, slot, or groove so that withdrawal cord 121 can pass by or through the annular gripping element. Alternatively, the withdrawal cord 121 can simply be slipped into a space arranged between the dilator 2 and the proximal part 650. In this case, fastening the proximal part 650 to the dilator 2 may have to be done in a discontinued fashion. The free end of the withdrawal cord 121 may be secured to stay outside of the dilator 2.

When a user wants to perform intromission of a tampon 3, the user takes hold of the cover element 605 used as a packaging bag. Next, using one hand, the user exerts traction on the distal part 651, while maintaining with the other hand either the free end of the proximal part 650, or the annular gripping element 10 through the proximal part 650. The distal part 651 is thus made to tear away at the areas of weakness 652, as represented in FIG. 26, and can afterwards be discarded. The proximal part 650 may be prefastened to the annular gripping element 10, or some other portion of the dilator, in a manner known to those skilled in the art, including by adhesion, bonding, and/or welding, or may be otherwise configured so that it continues to cover the tampon and proximal end of the dilator after the distal part 651 of the cover element 650 is removed.

Finally, the user can insert the tampon 3 into the vaginal cavity by pushing the proximal end of the tampon 3 through the proximal part 650, still being protected from fluids contact due to the proximal part 650 being between the pushing finger and the vaginal cavity.

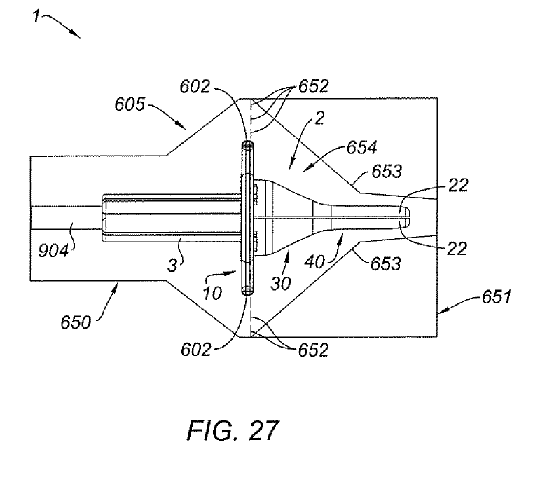

As depicted in FIG. 27, the proximal part 650 may be fitted with a pushing member 904. The proximal end of the pushing member 904 is attached to the proximal part 650 of cover element 605. The distal end of pushing member 904 may be placed in contact with the proximal end of tampon 3, so that the user may exert force on the proximal end of pushing member 904 to push tampon 3 through the dilator 2.

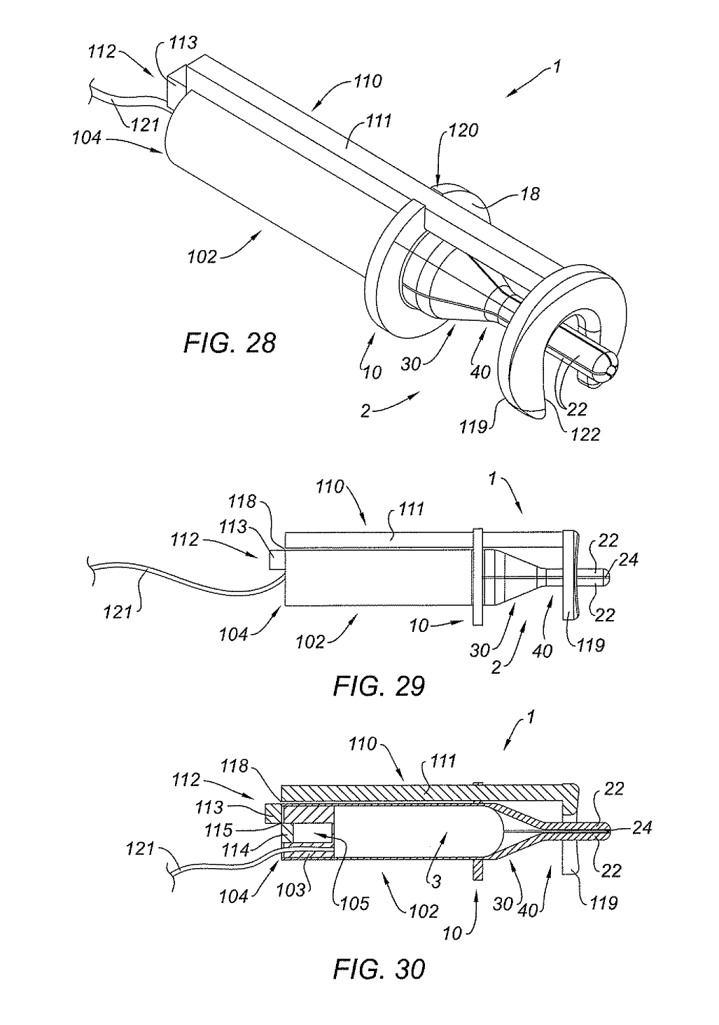

Referring now to FIG. 28, the applicator 1 may be configured with a dilator 2 as described above, the dilator 2 further comprising a tube 102 extending in a direction away from the distal end of the dilator 2, starting from the annular gripping element 10. As shown in FIGS. 28 to 30, tube 102 is cylindrical, with an interior diameter substantially equal to the diameter of the inner periphery of annular gripping element 10, and a length sufficient to allow tube 102 to completely house the tampon 3. However, the tube need not be limited to any particular length, diameter, or shape, as the tube may be configured in a variety of ways to achieve a number of functional and aesthetic purposes.

Referring to FIG. 30, after the tampon 3 is inserted into the tube 102, a ring 103 is slipped into the tube 102 by a proximal end area 104 thereof. This ring 103 has a longitudinal center housing 105 having a preferably polygonal, e.g., square, cross-section comprising an open proximal introducing face 106 and a closed distal end face 107. The proximal introducing face 106 has a corresponding cross-section, and is fitted with ribs 108 so as to make the opening area of the proximal introducing face 106 slightly smaller than that of the housing 105. The ring 103 further comprises longitudinal channels 109 disposed around the center housing 105.

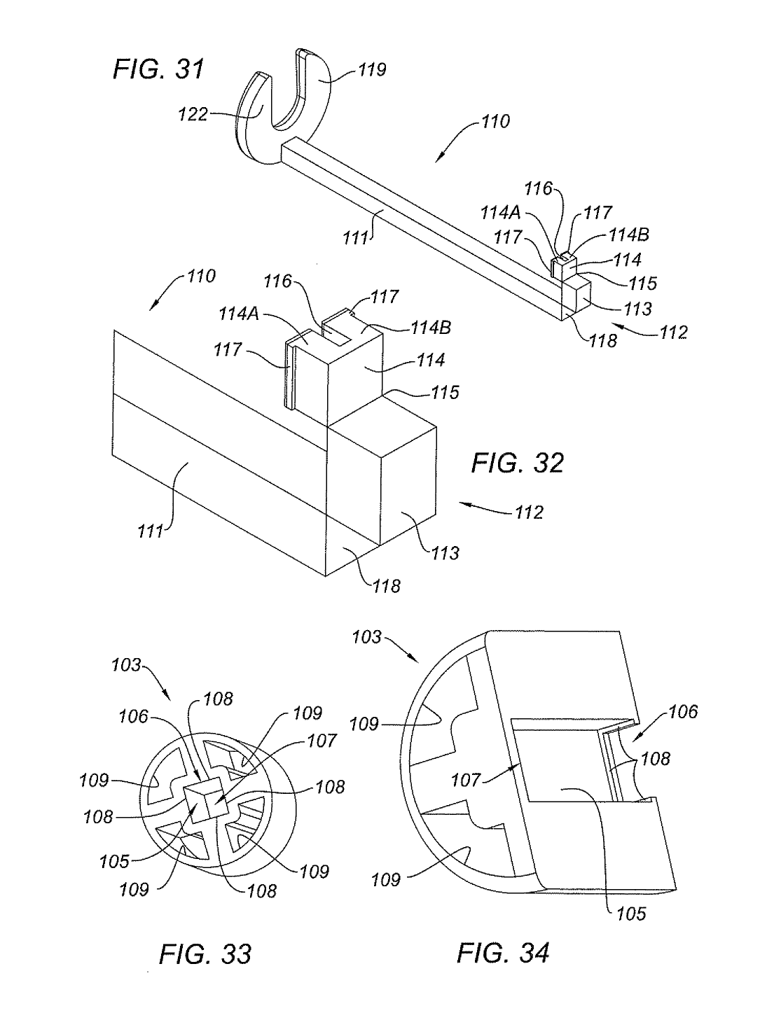

Prior to inserting the ring 103 into the tube 102, an integral pushing member 110 having a preferably square cross-section, comprising a plunger 111 hinged around a free end element 112, is inserted into the ring 103. This end element 112 is divided into a first block 113 and a second end block 114 disposed one after the other, and attached to each other by a material thread 115, as depicted in FIG. 31. The first block 113 and the second end block 114 can thus be driven by rotation with respect to each other.

The second end block 114 is divided into two symmetrical segments 114a, 114b by means of a slot 116, and each segment 114a, 114b comprises a contact surface 117 oriented outwards. The second end block 114 is shaped to have a cross-section, due to the two contact surfaces 117, that is slightly greater than that of the proximal introducing face 106 of the ring 103. The plunger 111 is also connected to the first block 113 by a material thread 118, so that the latter can thus be driven by rotation with respect to each other. At the opposite end thereof, the plunger 111 is fitted with a pushing element 119 comprising an opening 122.

Thus, the second end block 114 is forcibly inserted into the housing 105, with the segments 114a, 114b converging towards each other under the stress, and returning to their initial resting state after the contact surfaces 117 have moved past the ribs 108. The second end block 114 thus ends up being inserted inside the housing 105, unable to leave it, in as far as the ribs 108 act as a stop with regard to the contact surfaces 117. The first block 113 stays outside the tube 102, so that the plunger 111 can be folded back into the retracted position along the dilator 2, as shown in FIG. 28. The plunger 111 finally enters into the notch 120 of the gripping member 6 to be as close as possible to the dilator 2, and thus minimize the global volume of the assembly in the resting position.

Finally, the withdrawal cord 121 of the tampon 3 is positioned by means of an appropriate tool so that it passes one of the channels 109 and is thereby accessible from the outside of the assembly.

A user wishing to use the assembly starts by arming the pushing member 110 by deploying the plunger 111. For this purpose, the plunger 111 is rotated at an angle of about 180° around the material thread 118, and the first block 113 in turn is rotated around the material thread 115.

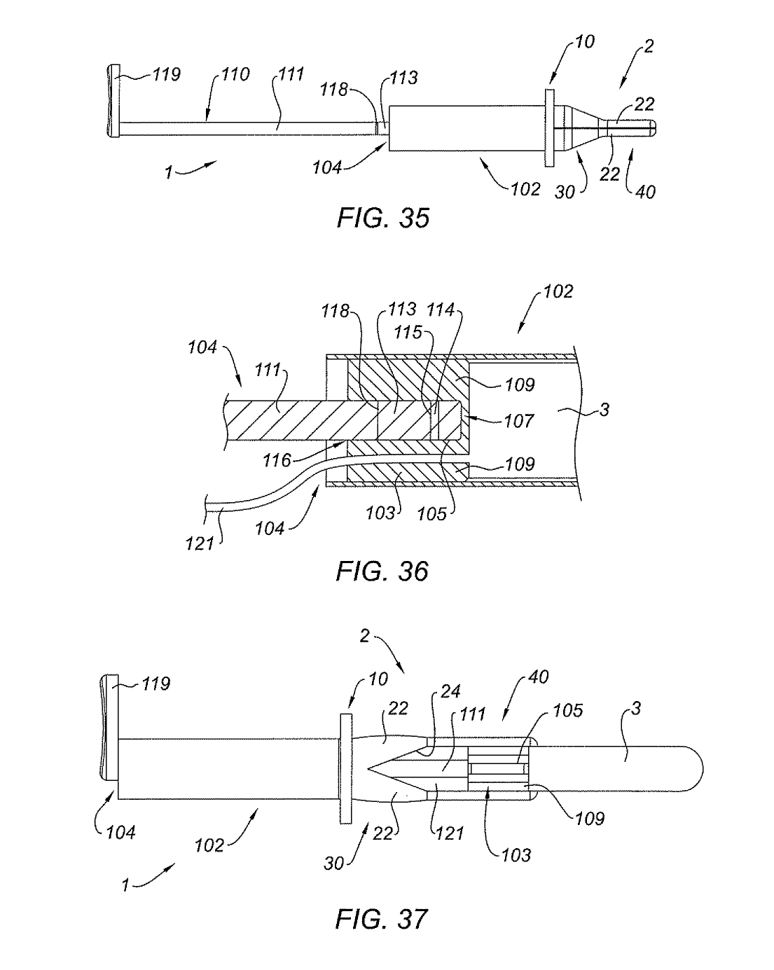

Once the pushing member 110 is completely deployed and the plunger ends up as an extension of the dilator 2, as represented in FIG. 35, the user can exert pressure on the pushing element 119. The pushing member 110 will slide until the second end block 114 comes into abutment against the distal end face 107 of the ring 103. At this time, the two material threads 115 and 118 end up inside the housing 105, as represented in FIG. 36, which prohibits any pivoting of the first block 113 with respect to the second end block 114, as well as any pivoting of the plunger 111 with respect to the first block 113. Thereby, the pushing member 110 is blocked in the maximum deployment position thereof by the housing 105 of the ring 103.

If sufficient force is exerted on the pushing element 119, the ring will be translated by the pushing member 110 along the tube 102, and will in turn force the tampon 3 to slide in the direction of the neck section 40. Extraction of the tampon 3 into the vaginal cavity is then done in a manner as already described before.

The ring 103 remains captive of the neck section 40 after the tampon 3 has been released under the effect of the longitudinal members 22 which tend to close, as represented in FIG. 37. Although tube 102 as depicted is cylindrical, the cross-sectional shape of the tube 102 may alternatively be, for example, rectangular, square, triangular, hexagonal, or any other polygonal shape, or any other shape comprising straight and/or curved lines. The shape of the ring 103 may also be adapted accordingly.

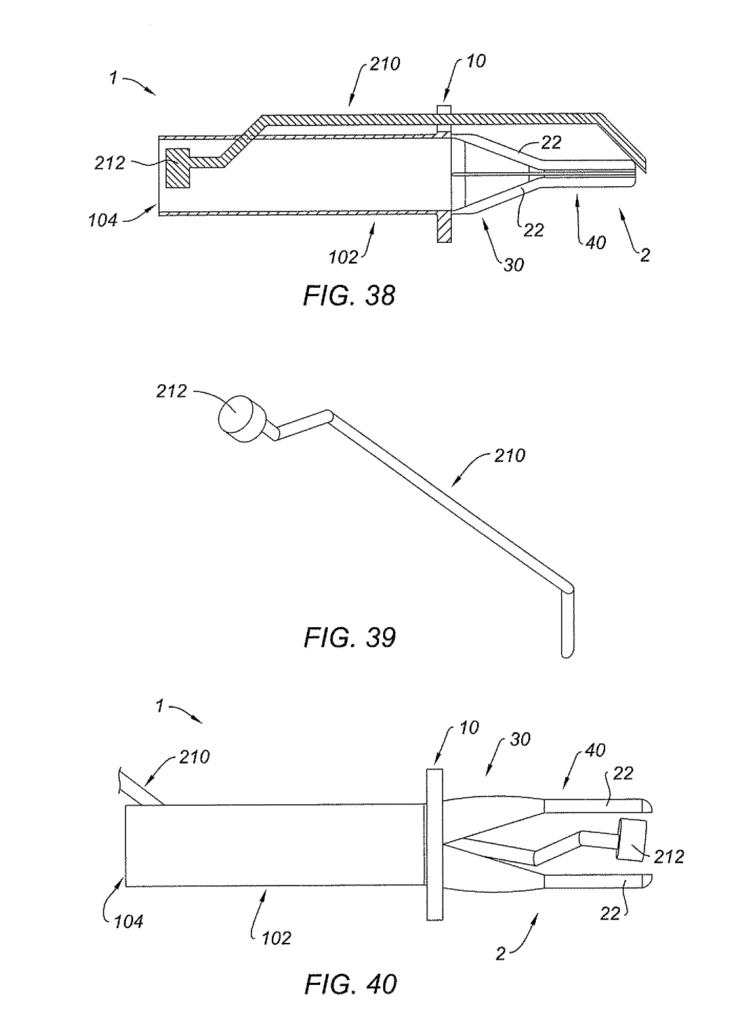

An alternative of this assembly that does not require a ring element is represented in FIGS. 38 to 40. The pushing member 210 is rigid, has a substantially bent shape, and has a preferably cylinder-shaped free end element 212. As before, the pushing member 210 is armed by pivoting it through an angle of about 180°. The proximal end area 104 of tube 102 is provided with a longitudinal slot (not shown) for receiving the pushing member 210 when it is in a retracted position. Extraction of the tampon 3 is done by translating the pushing member 210, so that the free end element 212 makes the tampon 3 slide along the dilator 2 until it is expelled into the vaginal cavity.

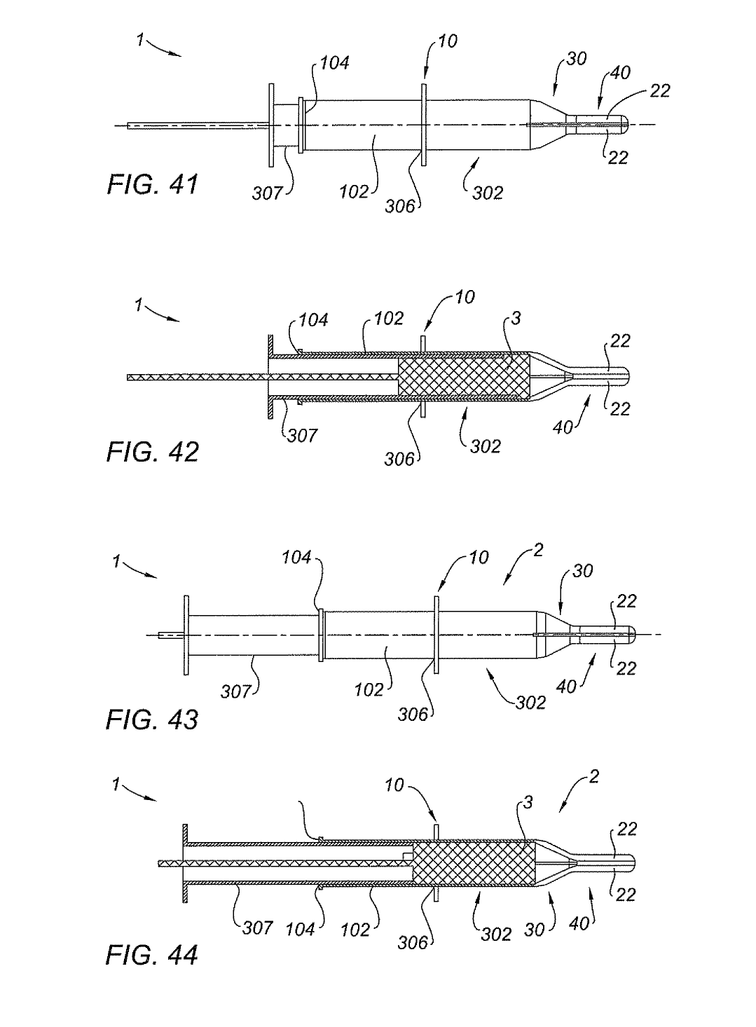

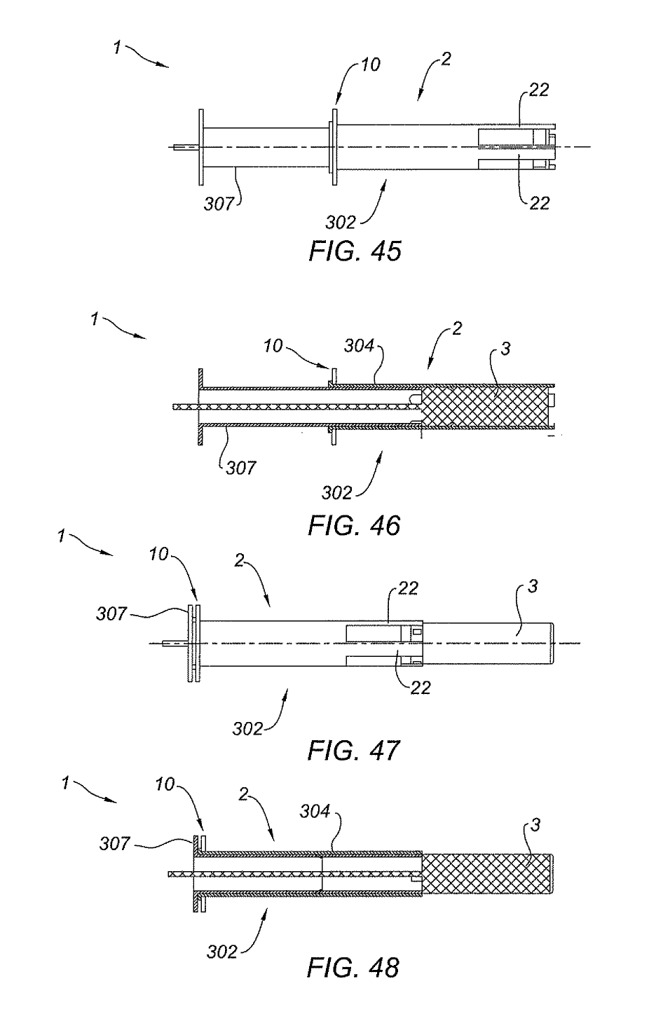

Alternative embodiments of an assembly are represented in FIGS. 41 to 48. In these embodiments, the dilator 2 further comprises an elongated tube section 302 between the conical section 30 and the annular gripping element 10 and optionally extending proximally past the gripping element 10. Although elongated tube section 302 as depicted is cylindrical, the cross-sectional shape of the elongated tube section 302 may alternatively be, for example, rectangular, square, triangular, hexagonal, or any other polygonal shape, or any other shape comprising straight and/or curved lines. The assembly comprises a tube 102 having a proximal end area 104 and a distal end 306.

A tampon 3 is inserted into the tube 102, then a plunger 307 is introduced into the proximal end area 104 of tube 102. The plunger 307 may be used to displace the tampon 3 along the tube 102 and the dilator 2. As shown in FIG. 42, the tampon 3 may be pre-loaded, for example, partially inside a hollow plunger 307. In such an embodiment, the plunger may be slid back behind the tampon 3 as shown in FIG. 44, allowing the tampon to expand and the plunger to abut its proximal end. The plunger 307 can then be depressed to intromit the tampon.

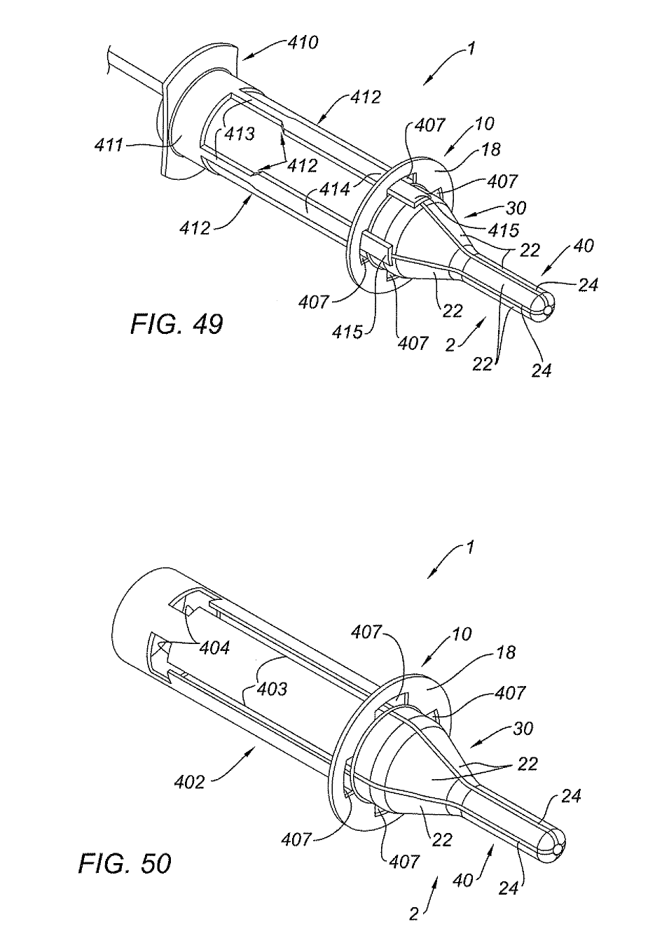

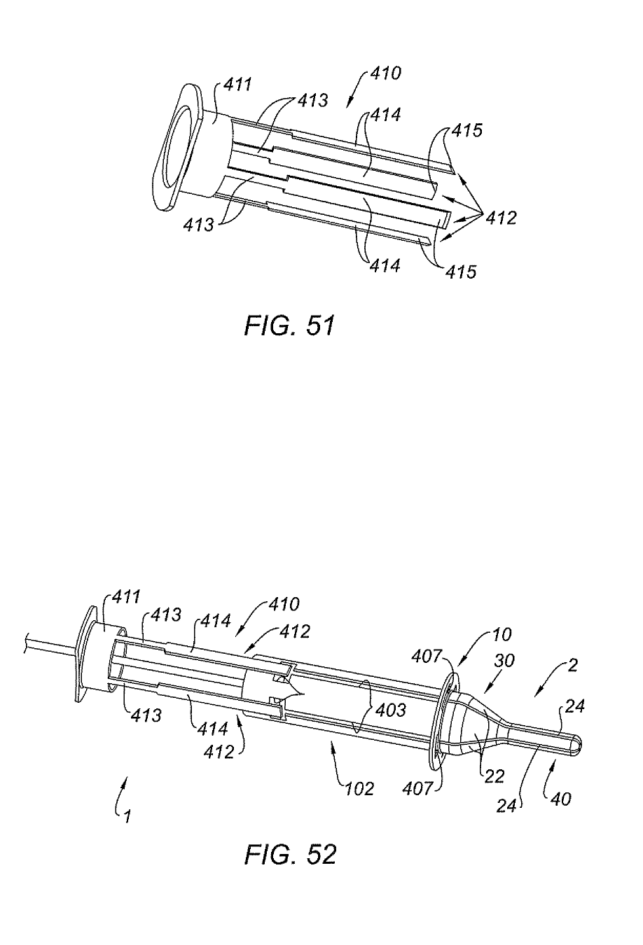

Another embodiment is shown in FIGS. 49 to 52. In this example, the tube 102 comprises four longitudinal slots 403 distributed around the circumference of the tube 102, each having an enlarged proximal end 404, and the annular gripping member 10 is configured with four orifices 407 disposed respectively as an extension of the four slots 403.

The assembly also comprises a hollow pushing member 410 divided into a base 411 extended by four longitudinal tabs 412 distributed around the circumference of hollow pushing member 410. Each tab 412 comprises a proximal part 413 having a reduced cross-section and a distal part 414 having an enlarged cross-section, the latter being chosen to be smaller than the cross-section of the proximal end 404, but greater than that of slot 403. This distal part 414 ends in a free end 415 curved inwards.

As depicted in FIG. 49, the pushing member 410 is previously positioned around the dilator 401. The tabs 412 have sufficient radial elasticity to enclose the dilator 2. Each free end 415 passes the corresponding orifice 407. A tampon (not shown) is inserted into the dilator 2.

A user wishing to use the assembly starts by arming the pushing member 410 by pulling the pushing member 410. During the translation, the tabs 412 slide on the slots 403 without being able to go through because of the dimensions of their respective cross-sections. At the end of arming, and as represented in FIG. 52, the free ends 415 of the tabs 412 emerge in the enlarged proximal end 404.

When the user exerts pressure on the base 411, the free ends 415 will first slide under the slots 403 because the tabs 412 tend to return to their resting position, then slide inside the proximal section 402 over the entire length of the slots 403. During this movement, the tabs 412 cannot leave the slots 403 because of the dimensions of their respective cross-sections.

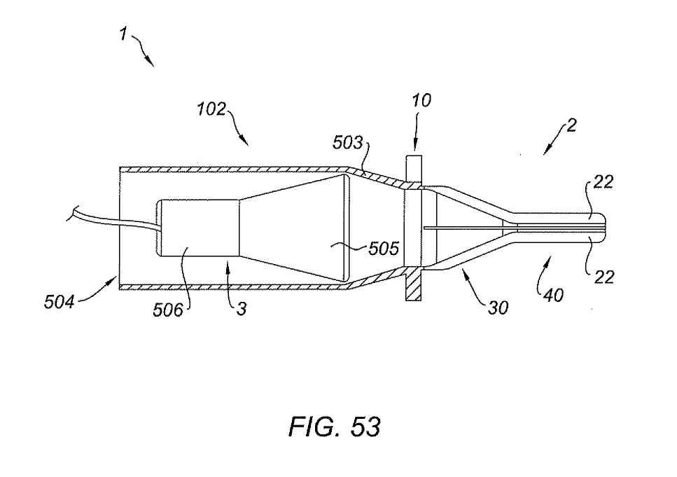

Referring to FIG. 53, tube 102 may be configured to have an enlarged diameter section 520 that comprises a substantial length of tube 102, and a tapered section 503 that tapers the cross-sectional area of tube 102 from the cross-sectional area of the enlarged diameter section 520 to the cross-sectional area of opening 52. This configuration makes it possible to use tampons that are not fully compressed and that have a diameter that is greater than the diameter of opening 52. In this case, the tapered section 503 functions to compress the tampon as it is being pushed through the enlarged diameter section 520 into the tapered section 503, so that the tampon is compressed to have a diameter adapted to pass through the interior passageway of the dilator. For example, a tampon having a diameter of 14 mm may be used instead of a conventional tampon having a diameter on the order of 12 mm.

Such a configuration also allows for the use of tampons that are not conventionally configured. For example, as shown in FIG. 53, the tampon 3 inserted into the tube 102 comprises a flared distal end 505 in the shape of a truncated cone, and a proximal end 506 having a conventional cross-section. A tampon with a flared distal end offers a greater contact surface area to absorb flow than that offered by a standard tampon. In addition, because the distal end of the tampon is not compressed, or at least not fully compressed, it is able to start absorbing flow almost immediately upon intromission into the vaginal cavity, whereas standard tampons may take up to a minute to start absorbing flow due to their highly compressed nature.

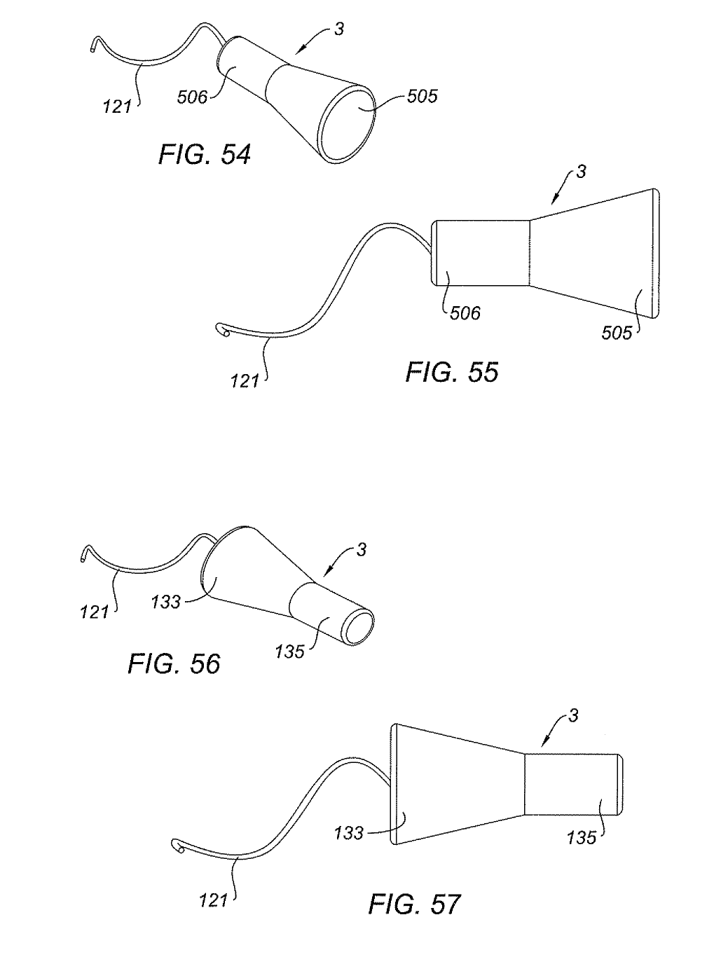

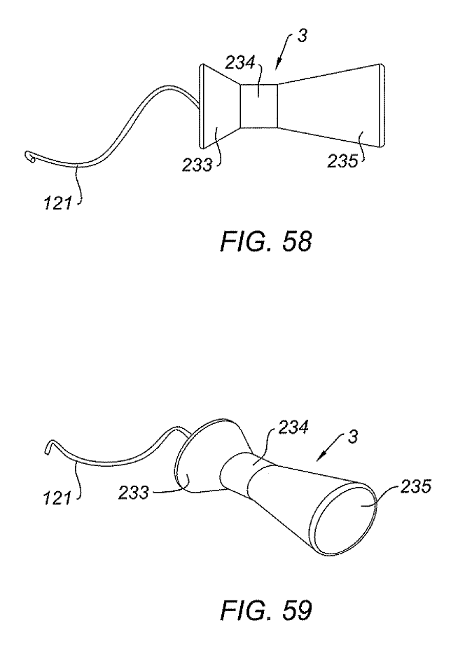

FIGS. 54 to 59 depict three exemplary configurations of tampons 3 that may be used with an assembly as represented in FIG. 53. The tampon 3 of FIGS. 54 and 55 corresponds to the one illustrated in FIG. 53. The tampon 3 represented in FIGS. 56 and 57 comprises a flared proximal end 133 in the shape of a truncated cone and a flat distal end 135. Such a tampon is advantageous in that it decreases the risk of downward leakage, and users having a larger than average vaginal cavity are less likely to lose it. The tampon 3 represented in FIGS. 58 and 59 has a flared proximal end 233 and a flared distal end 235, in the shape of a truncated cone and disposed on either side of a cylindrical center section 234. Such a configuration allows a combination of the advantages discussed before.

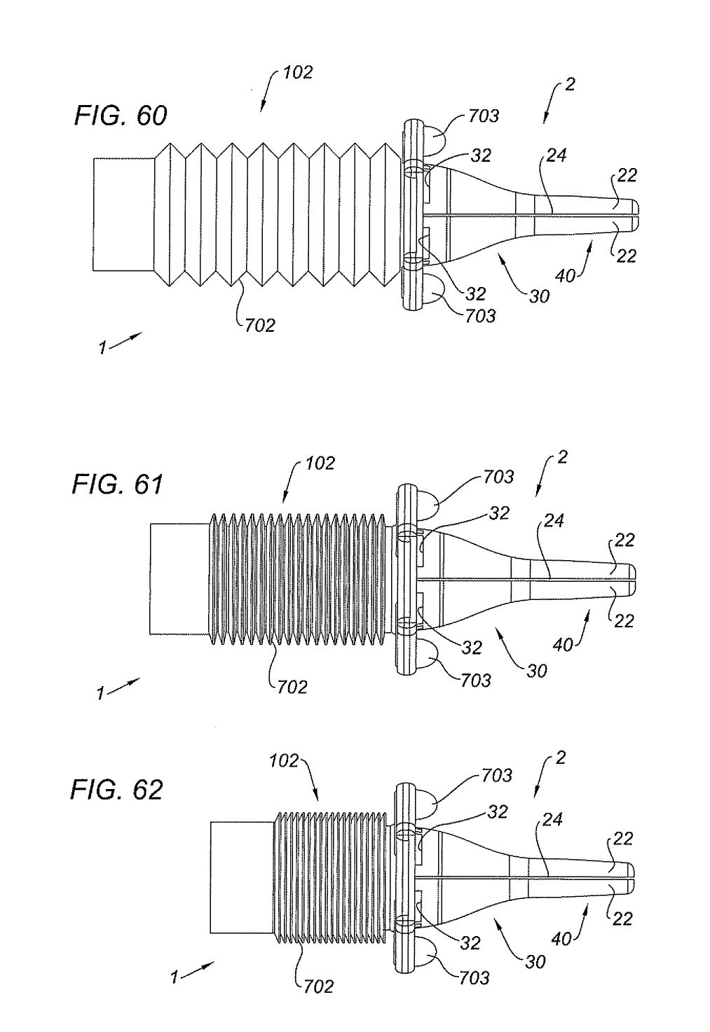

Referring to FIGS. 60 to 62, tube 102 may be configured to be compressible. For example, at least a portion of tube 102 may be configured with accordion pleats 702. When the user wishes to insert a tampon into her vaginal cavity, the tampon is inserted into tube 102, wherein tube 102 is in an elongated position, such as that shown in FIG. 60. By applying force to the proximal end of tube 102, accordion pleats 702 compress until tube 102 reaches a position of maximum compression, such as that depicted in FIG. 62.

When a pushing member (not depicted) is added to cause the displacement of the tampon inside the tube 102 and the dilator 2, using a compressible tube 702 allows for the overall length of the pushing member to be reduced by a distance equal to the difference of the length between the elongated position and the position of maximum compression of the longitudinal member 702.

It should be noted that for the sake of simplicity of the drawing, the longitudinal members 22 of the dilator 701 are represented only in the resting position. Obviously, these longitudinal members 22 would be in the opening position when the longitudinal member 702 is being compressed and the tampon has started to slide along the neck section 40.

In any of the embodiments discussed above, the opening 52 of the dilator 2 may be provided or fitted with a bushing member (not depicted) that narrows the opening size by which a tampon can be introduced. The bushing may be configured to provide additional support to a tampon introduced into the dilator 2, or to help guide the tampon more centrally to the interior passageway. The bushing may be permanently secured to the dilator, or be configured to be removable.

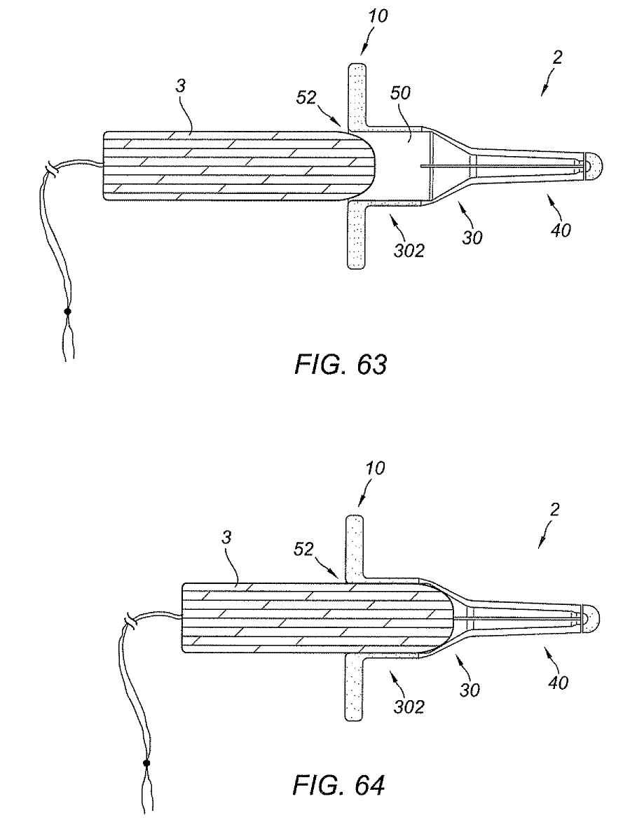

Referring to FIGS. 63 and 64, in addition to, or as an alternative to using a bushing member, the dilator 2 may further comprise an elongated tube section 302 between the conical section 30 and the annular gripping element 10 configured to provide additional support to a tampon introduced into the dilator 2, and/or to help guide the tampon more centrally and more parallel to the longitudinal axis of the interior passageway. Although elongated tube section 302 as depicted is cylindrical, the cross-sectional shape of the elongated tube section 302 may alternatively be, for example, rectangular, square, triangular, hexagonal, or any other polygonal shape, or any other shape comprising straight and/or curved lines.

Although the invention has been described in relation to specific exemplary embodiments, it is in no way limited thereto, and it comprises all the technical equivalents of the means described as well as the combinations thereof. Also, it should be noted that an applicator according to the disclosure could also be used for delivering any kind of body into one of orifices presented by the human body, such as for delivering rectal suppositories.

What is claimed is:

1. A tampon applicator, comprising:
an annular gripping element having an inner periphery defining an opening for insertion of a tampon;
a dilating element connected to the annular gripping element, the dilating element comprising a plurality of adjacent longitudinal members delimited by a plurality of longitudinal slots;
the plurality of adjacent longitudinal members forming:
an elongated neck at a distal end of the applicator, and
a hollow conical section having an external surface tapering distally inwardly and connected between the gripping element and the neck;
wherein insertion of a tampon through the opening and through the hollow conical section radially separates the longitudinal members to allow expulsion of the tampon distally between the longitudinal members.

2. The applicator of claim 1, wherein a taper angle of the external surface of the conical section is substantially greater than any taper angle of an external surface of the elongated neck.

3. The applicator of claim 2, wherein the elongated neck is substantially cylindrical.

4. The applicator of claim 1, wherein the elongated neck is longer than the conical section in a direction of an axis of the applicator.

5. The applicator of claim 1, wherein the longitudinal members are separated from each other by slots that extend longitudinally a distance into the conical section.

6. The applicator of claim 5, wherein the slots extend along an entire length of the conical section.

7. The applicator of claim 1, wherein the elongated neck at rest has a maximum external diameter of up to 5 mm.

8. The applicator of claim 7, wherein the elongated neck has a length of at least 1 cm.

9. The applicator of claim 1, wherein each longitudinal member has a distal tip that is substantially flush with distal tips of the other longitudinal members.

10. The applicator of claim 1, wherein a first longitudinal member has a tip that extends distally beyond tips of the other longitudinal members, the tip of the first elongated member having a geometry that covers the ends of the other tips.

11. The applicator of claim 1, further comprising a longitudinal groove formed on an exterior surface of at least one said longitudinal member.

12. The applicator of claim 5, further comprising a junction point connecting two adjacent longitudinal members, the junction point traversing a longitudinal slot.

13. The applicator of claim 5, wherein the adjacent longitudinal members are connected by two junction points.

14. The applicator of claim 1, wherein the conical section has an interior surface defining a portion of an interior passageway having a substantially decreasing cross-sectional area in a distal direction.

15. The applicator of claim 1, wherein the conical section has an interior surface defining an area of rugosity.

16. The applicator of claim 4, wherein the interior surface defining an area of rugosity is configured in a series of internal shoulders each having a substantially circular edge.

17. The applicator of claim 5, wherein it comprises four longitudinal slots evenly distributed about the circumference of the dilating element.

18. The applicator of claim 1, wherein at least one longitudinal member comprises a window.

19. The applicator of claim 1, further comprising a guiding device for pushing a tampon through the applicator.

20. The applicator of claim 19, wherein the guiding device comprises a spring and a pushing member attached to the spring.

21. The applicator of claim 20, further comprising a plunger, a portion of the plunger being in slidable communication with an interior portion of the pushing member.

22. The applicator of claim 1, further comprising a cover element configured to be positionable over a proximal end of a tampon after a distal end of the tampon is situated within an interior passageway of the applicator.

23. The applicator of claim 22, wherein the cover element comprises a substantially rectangular band of material, attachable to the applicator.

24. The applicator of claim 22, wherein:
the applicator contains a tampon; and
the cover element is a sleeve enveloping the tampon and applicator.

25. The applicator of claim 24, wherein the sleeve comprises a proximal part and a distal part, and an area of weakness configured so that the distal part of the sleeve may be detached from the proximal part of the sleeve.

26. The applicator of claim 25, wherein the proximal part of the sleeve is secured to the applicator.

27. The applicator of claim 25, wherein a portion of the distal part of the sleeve is conformed to an exterior shape of the dilating element.

28. The applicator of claim 27, wherein the distal part of the sleeve impedes radial spreading of the longitudinal members of the dilating element.

29. The applicator of claim 25, further comprising a pushing member attached to an interior surface of the proximal part of the sleeve.

30. The applicator of claim 1, further comprising a tube attached to the annular gripping element proximally of the conical section.

31. The applicator of claim 30, further comprising:
a pushing member configured to move from a retracted position, in which the pushing member is substantially disposed along the applicator, to an activating position, in which the pushing member is disposed as an extension of the applicator.

32. The applicator of claim 31, wherein the pushing member comprises a plunger hinged around a free end element introduced into a proximal end area of the tube.

33. The applicator of claim 32, wherein the plunger and the end element are configured to be able to be driven by rotation with respect to each other.

34. The applicator of claim 31, further comprising a ring insertable into a proximal end area of the tube, the ring being configured with a housing for receiving the end element of the pushing member.

35. The applicator of claim 30, wherein a portion of the tube has an interior surface defining a passageway tapering radially inwardly in a distal direction.

36. The applicator of claim 31, wherein:
the pushing member has a substantially bent shape, and a cylinder-shaped free end element; and
a portion of a proximal end of the tube is configured with a longitudinal slot for receiving the pushing member when the pushing member is in the retracted position.

37. The applicator of claim 32, wherein:
the tube comprises four longitudinal slots evenly distributed around the circumference of the tube, each slot having an enlarged proximal end;
the annular gripping element is configured with four orifices disposed respectively as an extension of the four slots;
the applicator further comprises a hollow pushing member having a base extended by four longitudinal members uniformly distributed around a circumference of the hollow pushing member; and
each pushing member extension comprises a proximal part having a reduced cross-section and a distal part having an enlarged cross-section that is smaller than the cross-section of the proximal end of the tube slot, but greater than the cross-section of an other portion of the tube slot.

38. The applicator of claim 30, wherein:
a first portion of the tube interior passageway has a cross-sectional area greater than the cross-sectional area of the opening defined by the inner periphery of the annular gripping element; and
a second portion of the tube interior passageway decreases in cross-sectional area in the direction of the neck section, the second portion being between the first portion and the annular gripping element.

39. The applicator of claim 30, wherein the tube is configured to be compressible in a longitudinal direction towards the annular gripping element.

40. The applicator of claim 39, wherein the tube is configured with accordion pleats.

41. The applicator of claim 1, further comprising:
a tube connected to the annular gripping element and the conical section; and
a hollow plunger configured to be slidably receivable into a proximal end of the tube.
